(19) United States
(12) Patent Application Publication
(10) Pub. No.: US 2006/0182994 A1
(43) Pub. Date: Aug. 17, 2006

(54) Anthracene derivative, organic electroluminescent device, and display unit

(76) Inventors: Yukinari Sakamoto, Tokyo (JP); Yoshihisa Miyabayashi, Kanagawa (JP); Shinichiro Tamura, Kanagawa (JP); Naoyuki Ueda, Kanagawa (JP); Kenji Ueda, Kanagawa (JP); Tadahiko Yoshinaga, Kanagawa (JP)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/329,647

(22) Filed: Jan. 11, 2006

(30) Foreign Application Priority Data
Jan. 18, 2005 (JP) P2005-009981
Jan. 21, 2005 (JP) P2005-013517

(51) Int. Cl. H01L 51/54; H05B 33/14; C09K 11/06; C07C 211/00

(52) U.S. Cl. 428/690; 428/917; 313/504; 313/506; 257/88; 257/E51.051; 564/433; 564/434; 564/429; 548/440; 540/588

(57) ABSTRACT

An anthracene derivative with general formula (1) is provided: 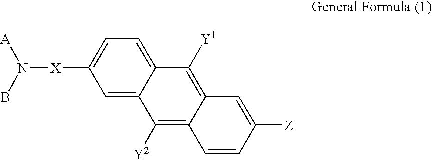
wherein X represents a substituted or unsubstituted C6-28 arylene group, or a substituted or unsubstituted C5-21 divalent heterocyclic group; A and B each independently represent a substituted or unsubstituted C1-20 alkyl group, a substituted or unsubstituted C6-28 aryl group, or a substituted or unsubstituted C5-21 heterocyclic group, and A and B may be bonded together to form a ring; Y¹ and Y² each independently represent a hydrogen atom, a substituted or unsubstituted C1-20 alkyl group or a C1-20 alkoxy group; and Z represents a substituted or unsubstituted C6-30 aryl group atoms, a substituted or unsubstituted C5-21 heterocyclic group, a hydrogen atom, a substituted or unsubstituted C1-20 alkyl group, or a C1-20 alkoxy group.

### CROSS REFERENCES TO RELATED APPLICATIONS

The present invention contains subject matter related to Japanese Patent Applications JP 2005-009981 filed in the Japanese Patent Office on Jan. 18, 2005 and JP 2005-013517 filed in the Japanese Patent Office on Jan. 21, 2005, the entire contents of which are incorporated herein by reference.

### BACKGROUND OF THE INVENTION

1. Field of the Invention

The present invention relates to an anthracene derivative suitable for use as an organic material for organic electroluminescent devices, an organic electroluminescent device using the anthracene derivative, and a display unit including the organic electroluminescent device.

2. Description of the Related Art

Recently, display units including organic electroluminescent devices (i.e., organic EL devices) have become popular as self-emitting flat-panel displays which consume low power and have a high response rate and a wide view angle.

An organic electroluminescent device includes an organic layer sandwiched between a cathode and an anode, the organic layer containing an organic luminescent material which emits light in the presence of an applied current. As the organic layer, for example, a structure in which a hole-transport layer, a luminescent layer containing an organic luminescent material, and an electron-transport layer disposed in that order on an anode, or a structure in which a luminescent material is incorporated into an electron-transport layer to form a luminescent layer having an electron-transport property has been developed.

In case a display unit is fabricated using the organic electroluminescent device, one of the most important tasks is to ensure longer lifetime and reliability of the organic electroluminescent device. Under these circumstances, studies have been conducted on organic materials constituting organic electroluminescent devices.

Above all, with respect to materials having an anthracene skeleton, many derivatives, such as anthracene derivatives and bisanthracene derivatives containing an amino group or an aryl group, and anthracene derivatives containing a styryl group, have been studied. For example, refer to Japanese Unexamined Patent Application Publication Nos. 2003-146951, 9-268284, 9-268283, 2004-67528, and 2001-284050 (Patent Documents 1 to 5).

In particular, further improvement is required for blue luminescent materials in terms of color purity, luminous efficiency, and luminous lifetime. Studies based on stilbene, styrylarylene, or anthracene derivatives for example have been made so far. Refer to, for example, Materials Science and Engineering: R: Reports Volume 39, Issues 5-6, pp. 143-222, 2002 (Non-Patent Document 1) and Applied Physics Letters (U.S.), Vol. 67, No. 26, 1995, pp. 3853-3855 (Non-Patent Document 2).

### SUMMARY OF THE INVENTION

However, a blue luminescent material with higher luminous efficiency, longer lifetime, and higher color purity is desired.

According to an embodiment of the present invention, a material that is suitable for use as an organic material constituting an organic electroluminescent device is represented by general formula (1): 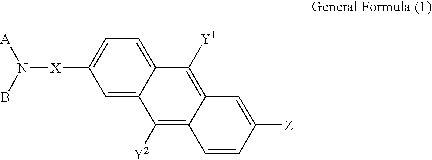
wherein X represents a substituted or unsubstituted arylene group having 6 to 28 carbon atoms, or a substituted or unsubstituted divalent heterocyclic group having 5 to 21 carbon atoms; A and B each independently represent a substituted or unsubstituted alkyl group having 1 to 20 carbon atoms, a substituted or unsubstituted aryl group having 6 to 28 carbon atoms, or a substituted or unsubstituted heterocyclic group having 5 to 21 carbon atoms, and A and B may be bonded together to form a ring; Y¹ and Y² each independently represent a hydrogen atom, a substituted or unsubstituted alkyl group having 1 to 20 carbon atoms, or an alkoxy group having 1 to 20 carbon atoms; and Z represents a substituted or unsubstituted aryl group having 6 to 30 carbon atoms, a substituted or unsubstituted heterocyclic group having 5 to 21 carbon atoms, a hydrogen atom, a substituted or unsubstituted alkyl group having 1 to 20 carbon atoms, or an alkoxy group having 1 to 20 carbon atoms. Here, the alkyl group includes a linear, branched, or cyclic alkyl group.

According to another embodiment of the present invention, a material that is suitable for use as an organic material constituting an organic electroluminescent device is represented by general formula (2): 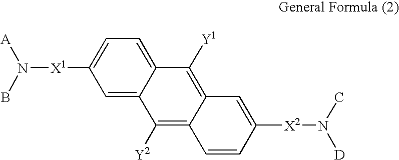
wherein X¹ and X² each independently represent a substituted or unsubstituted arylene group having 6 to 28 carbon atoms, or a substituted or unsubstituted divalent heterocyclic group having 5 to 21 carbon atoms; A, B, C, and D each independently represent a substituted or unsubstituted alkyl group having 1 to 20 carbon atoms, a substituted or unsubstituted aryl group having 6 to 28 carbon atoms, or a substituted or unsubstituted heterocyclic group having 5 to 21 carbon atoms, and A and B may be bonded together to form a ring and/or C and D may be bonded together to form a ring; and Y¹ and Y² each independently represent a hydrogen atom, a substituted or unsubstituted alkyl group having 1 to 20 carbon atoms, or an alkoxy group having 1 to 20 carbon atoms.

The anthracene derivative represented by general formula (1) or (2) is used for an organic layer of an organic electroluminescent device, and in particular, preferably used as a blue luminescent material.

### BRIEF DESCRIPTION OF THE DRAWINGS

FIG. 1 is a schematic sectional view of an organic electroluminescent device according to an embodiment of the present invention;

FIG. 2 is a graph showing fluorescence absorption spectrum of a compound (17) in a dioxane solution;

FIG. 3 is a graph showing fluorescence absorption spectrum of a compound (45) in a dioxane solution;

FIG. 4 is a graph showing fluorescence absorption spectrum of a compound (46) in a dioxane solution;

FIG. 5 is a graph showing fluorescence absorption spectrum of a compound (49) in a dioxane solution;

FIG. 6 is a graph showing fluorescence absorption spectrum of a compound (60) in a dioxane solution; and

FIG. 7 is a graph showing fluorescence absorption spectrum of a compound (73) in a dioxane solution.

### DESCRIPTION OF THE PREFERRED EMBODIMENTS

Embodiments of the present invention will be described below.

Anthracene Derivative

Specific examples of an anthracene derivative represented by general formula (1) or (2) according to an embodiment of the present invention will be described below.

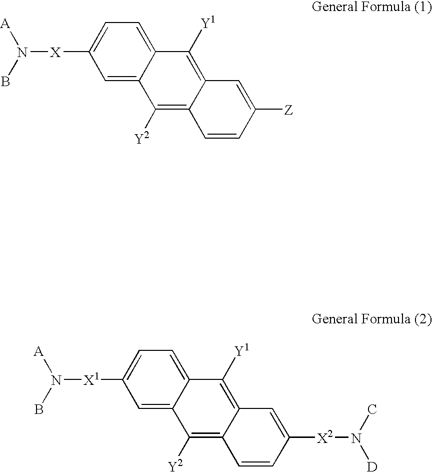

In general formulae (1) and (2), X, X¹, and X² each independently represent (a) a substituted or unsubstituted arylene group having 6 to 28 carbon atoms, or (b) a substituted or unsubstituted divalent heterocyclic group having 5 to 21 carbon atoms.

Among these, examples of the arylene group (a) include phenylene and divalent groups derived from aromatic hydrocarbons, such as biphenyl, terphenyl, naphthalene, anthracene, phenanthrene, pyrene, fluorene, fluoranthene, benzofluoranthene, dibenzofluoranthene, acephenanthrylene, aceanthrylene, triphenylene, acenaphthotriphenylene, chrysene, perylene, benzochrysene, naphthacene, pleiadene, picene, pentaphene, pentacene, tetraphenylene, trinaphthylene, benzophenanthrene, dibenzonaphthacene, benzoanthracene, dibenzoanthracene, benzonaphthacene, naphthopyrene, benzopyrene, dibenzopyrene, benzocyclooctene, anthranaphthacene, and acenaphthofluoranthene.

Furthermore, the arylene group (a) may be a divalent group derived from any combination of these aromatic hydrocarbons.

The substitution site of the arylene group (a) is not particularly limited. In order to achieve blue luminescence with higher color purity, preferably, the number of carbon atoms of the aromatic hydrocarbon directly bonded to the nitrogen atom is 6 to 18, and more preferably, the number of carbon atoms of the aromatic hydrocarbon directly bonded to the nitrogen atom is 6 to 14.

Examples of the heterocyclic group (b) include divalent groups derived from thiophene, benzothiophene, oxazole, benzooxazole, oxadiazole, pyridine, pyrimidine, pyrazine, quinoline, benzoquinoline, dibenzoquinoline, isoquinoline, benzisoquinoline, quinazoline, quinoxaline, acridine, phenanthridine, phenazine, phenoxazine, etc. and any combination of these compounds.

The substitution site of the heterocyclic group (b) is not particularly limited. In order to achieve blue luminescence with higher color purity, preferably, the number of carbon atoms of the heterocyclic group directly bonded to the nitrogen atom is 5 to 17, and more preferably, the number of carbon atoms of the heterocyclic group directly bonded to the nitrogen atom is 5 to 13.

In general formulae (1) and (2), X, X¹, and X² each may be a divalent group in which the arylene group (a) and the heterocyclic group (b), which are exemplified above, are bonded to each other.

Examples of the substituent for the arylene group (a) or the heterocyclic group (b) include a halogen atom, a hydroxyl group, a substituted or unsubstituted amino group, a substituted or unsubstituted alkyl group, a substituted or unsubstituted alkenyl group, a substituted or unsubstituted cycloalkyl group, a substituted or unsubstituted alkoxy group, a substituted or unsubstituted aromatic hydrocarbon group, a substituted or unsubstituted aromatic heterocyclic group, a substituted or unsubstituted aralkyl group, a substituted or unsubstituted aryloxy group, a substituted or unsubstituted alkoxycarbonyl group, and carboxyl group. The substitution site of the condensed ring and the number of substitutions are not particularly limited. Examples of the halogen atom include fluorine, chlorine, bromine, and iodine.

A and B in general formula (1) and A, B, C, and D in general formula (2) each independently represent (c) a substituted or unsubstituted alkyl group having 1 to 20 carbon atoms, (d) a substituted or unsubstituted aryl group having 6 to 28 carbon atoms, or (e) a substituted or unsubstituted heterocyclic group having 5 to 21 carbon atoms.

Among these, the alkyl group (c) may be linear, branched, or cyclic. Examples of the alkyl group include methyl, ethyl, propyl, isopropyl, n-butyl, s-butyl, isobutyl, tert-butyl, n-pentyl, n-hexyl, n-heptyl, n-octyl, hydroxymethyl, 1-hydroxyethyl, 2-hydroxyethyl, 2-hydroxyisobutyl, 1,2-dihydroxyethyl, 1,3-dihydroxyisopropyl, 2,3-dihydroxy-tert-butyl, 1,2,3-trihydroxypropyl, chloromethyl, 1-chloroethyl, 2-chloroethyl, 2-chloroisobutyl, 1,2-dichloroethyl, 1,3-dichloroisopropyl, 2,3-dichloro-tert-butyl, 1,2,3-trichloropropyl, bromomethyl, 1-bromoethyl, 2-bromoethyl, 2-bromoisobutyl, 1,2-dibromoethyl, 1,3-dibromoisopropyl, 2,3-dibromo-tert-butyl, 1,2,3-tribromopropyl, iodomethyl, 1-iodoethyl, 2-iodoethyl, 2-iodoisobutyl, 1,2-diiodoethyl, 1,3-diiodoisopropyl, 2,3-diiodo-tert-butyl, 1,2,3-triiodopropyl, aminomethyl, 1-aminoethyl, 2-aminoethyl, 2-aminoisobutyl, 1,2-diaminoethyl, 1,3-diaminoisopropyl, 2,3-diamino-tert-butyl, 1,2,3-triaminopropyl, cyanomethyl, 1-cyanoethyl, 2-cyanoethyl, 2-cyanoisobutyl, 1,2-dicyanoethyl, 1,3-dicyanoisopropyl, 2,3-dicyano-tert-butyl, 1,2,3-tricyanopropyl, nitromethyl, 1-nitroethyl, 2-nitroethyl, 2-nitroisobutyl, 1,2-dinitroethyl, 1,3-dinitroisopropyl, 2,3-dinitro-tert-butyl, 1,2,3-trinitropropyl, cyclopropyl, cyclobutyl, cyclopentyl, cyclohexyl, and 4-methylcyclohexyl.

Examples of the aryl group (d) include a phenyl group and monovalent groups derived from aromatic hydrocarbons, such as biphenyl, terphenyl, naphthalene, anthracene, phenanthrene, pyrene, fluorene, fluoranthene, benzofluoranthene, dibenzofluoranthene, acephenanthrylene, aceanthrylene, triphenylene, acenaphthotriphenylene, chrysene, perylene, benzochrysene, naphthacene, pleiadene, picene, pentaphene, pentacene, tetraphenylene, trinaphthylene, benzophenanthrene, dibenzonaphthacene, benzoanthracene, dibenzoanthracene, benzonaphthacene, naphthopyrene, benzopyrene, dibenzopyrene, benzocyclooctene, anthranaphthacene, and acenaphthofluoranthene.

Furthermore, the aryl group (d) may be a monovalent group derived from any combination of these aromatic hydrocarbons.

The substitution site of the aryl group (d) is not particularly limited. In order to achieve blue luminescence with higher color purity, preferably, the number of carbon atoms of the aromatic hydrocarbon directly bonded to the nitrogen atom is 6 to 18, and more preferably, the number of carbon atoms of the aromatic hydrocarbon directly bonded to the nitrogen atom is 6 to 14.

Examples of the heterocyclic group (e) include monovalent groups derived from thiophene, benzothiophene, oxazole, benzooxazole, oxadiazole, pyridine, pyrimidine, pyrazine, quinoline, benzoquinoline, dibenzoquinoline, isoquinoline, benzisoquinoline, quinazoline, quinoxaline, acridine, phenanthridine, phenazine, phenoxazine, etc. and any combination of these compounds.

The substitution site of the heterocyclic group (e) is not particularly limited. In order to achieve blue luminescence with higher color purity, preferably, the number of carbon atoms of the heterocyclic group directly bonded to the nitrogen atom is 5 to 17, and more preferably, the number of carbon atoms of the heterocyclic group directly bonded to the nitrogen atom is 5 to 13.

Furthermore, A and B in general formula (1) and A, B, C, and D in general formula (2) each may be a monovalent group in which the aryl group (d) and the heterocyclic group (e) are bonded to each other.

Examples of the substituent for the aryl group (d) or the heterocyclic group (e) include a halogen atom, a hydroxyl group, a substituted or unsubstituted amino group, a substituted or unsubstituted alkyl group, a substituted or unsubstituted alkenyl group, a substituted or unsubstituted cycloalkyl group, a substituted or unsubstituted alkoxy group, a substituted or unsubstituted aromatic hydrocarbon group, a substituted or unsubstituted aromatic heterocyclic group, a substituted or unsubstituted aralkyl group, a substituted or unsubstituted aryloxy group, a substituted or unsubstituted alkoxycarbonyl group, and a carboxyl group. The substitution site of the condensed ring and the number of substitutions are not particularly limited. Examples of the halogen atom include fluorine, chlorine, bromine, and iodine.

Introduction of a moderately bulky substituent into any of A and B in general formula (1) and A, B, C, and D in general formula (2) is effective in controlling the crystallization and suppressing bimolecular excitation, which are related to device characteristics, and luminous efficiency and luminous lifetime can be further improved. Therefore, it is preferable to introduce a substituent selected from an alkyl group, an alkoxy group, an alkenyl group, a heterocyclic group, and an aryl group into the aryl group (d) or the heterocyclic group (e).

In addition, if A and B in general formula (1) or A and B, and C and D each are bonded together by a single bond, a carbon ring bond, or the like, the compound has an improved glass transition temperature and excellent heat resistance.

Furthermore, in general formulae (1) and (2), Y¹ and Y² each independently represent (f) a hydrogen atom, (g) a substituted or unsubstituted alkyl group having 1 to 20 carbon atoms, or (h) an alkoxy group having 1 to 20 carbon atoms.

Among these, the alkyl group is the same as the alkyl group (c) in A and B described above.

The alkoxy group (h) is represented by —OR, and examples of R include methyl, ethyl, propyl, isopropyl, n-butyl, s-butyl, isobutyl, tert-butyl, n-pentyl, n-hexyl, n-heptyl, n-octyl, hydroxymethyl, 1-hydroxyethyl, 2-hydroxyethyl, 2-hydroxyisobutyl, 1,2-dihydroxyethyl, 1,3-dihydroxyisopropyl, 2,3-dihydroxy-tert-butyl, 1,2,3-trihydroxypropyl, chloromethyl, 1-chloroethyl, 2-chloroethyl, 2-chloroisobutyl, 1,2-dichloroethyl, 1,3-dichloroisopropyl, 2,3-dichloro-tert-butyl, 1,2,3-trichloropropyl, bromomethyl, 1-bromoethyl, 2-bromoethyl, 2-bromoisobutyl, 1,2-dibromoethyl, 1,3-dibromoisopropyl, 2,3-dibromo-tert-butyl, 1,2,3-tribromopropyl, iodomethyl, 1-iodoethyl, 2-iodoethyl, 2-iodoisobutyl, 1,2-diiodoethyl, 1,3-diiodoisopropyl, 2,3-diiodo-tert-butyl, 1,2,3-triiodopropyl, aminomethyl, 1-aminoethyl, 2-aminoethyl, 2-aminoisobutyl, 1,2-diaminoethyl, 1,3-diaminoisopropyl, 2,3-diamino-tert-butyl, 1,2,3-triaminopropyl, cyanomethyl, 1-cyanoethyl, 2-cyanoethyl, 2-cyanoisobutyl, 1,2-dicyanoethyl, 1,3-dicyanoisopropyl, 2,3-dicyano-tert-butyl, 1,2,3-tricyanopropyl, nitromethyl, 1-nitroethyl, 2-nitroethyl, 2-nitroisobutyl, 1,2-dinitroethyl, 1,3-dinitroisopropyl, 2,3-dinitro-tert-butyl, and 1,2,3-trinitropropyl.

In general formula (1), Z represents (i) a substituted or unsubstituted aryl group having 6 to 30 carbon atoms, (j) a substituted or unsubstituted heterocyclic group having 5 to 21 carbon atoms, (k) a hydrogen atom, (l) a substituted or unsubstituted alkyl group having 1 to 20 carbon atoms, or (m) an alkoxy group having 1 to 20 carbon atoms.

Among these, the aryl group (i) is the same as the aryl group (d) in A and B described above except that the number of carbon atoms is 6 to 30. The heterocyclic group (j) is the same as the heterocyclic group (e) in A and B described above. The alkyl group (l) is the same as the alkyl group (c) in A and B described above. The alkoxy group (m) is the same as the alkoxy group (h) in Y¹ and Y² described above.

Compounds (1) to (44) will be shown below as the specific examples of the anthracene derivative represented by general formula (1). However, it is to be understood that the present invention is not limited thereto.

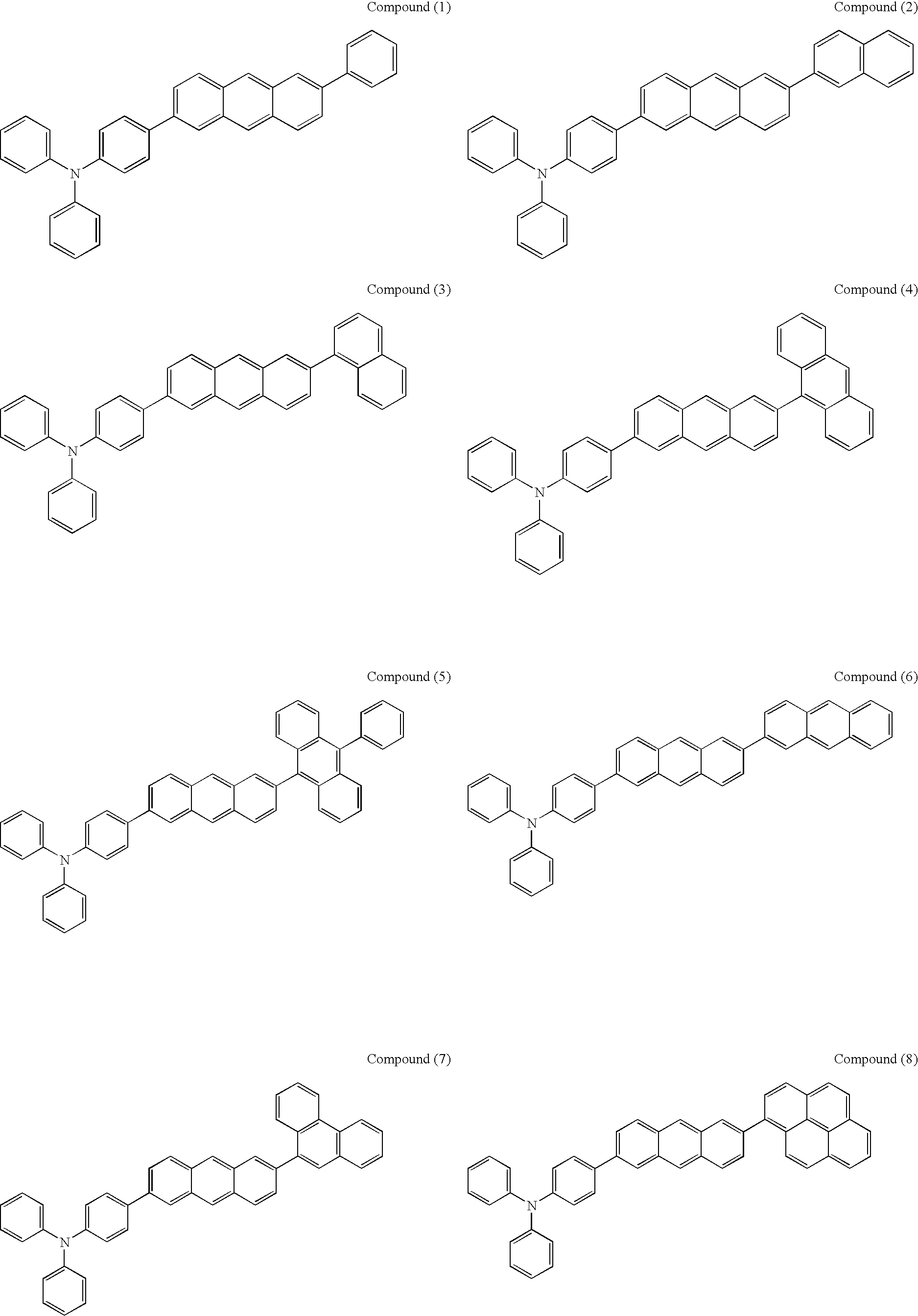

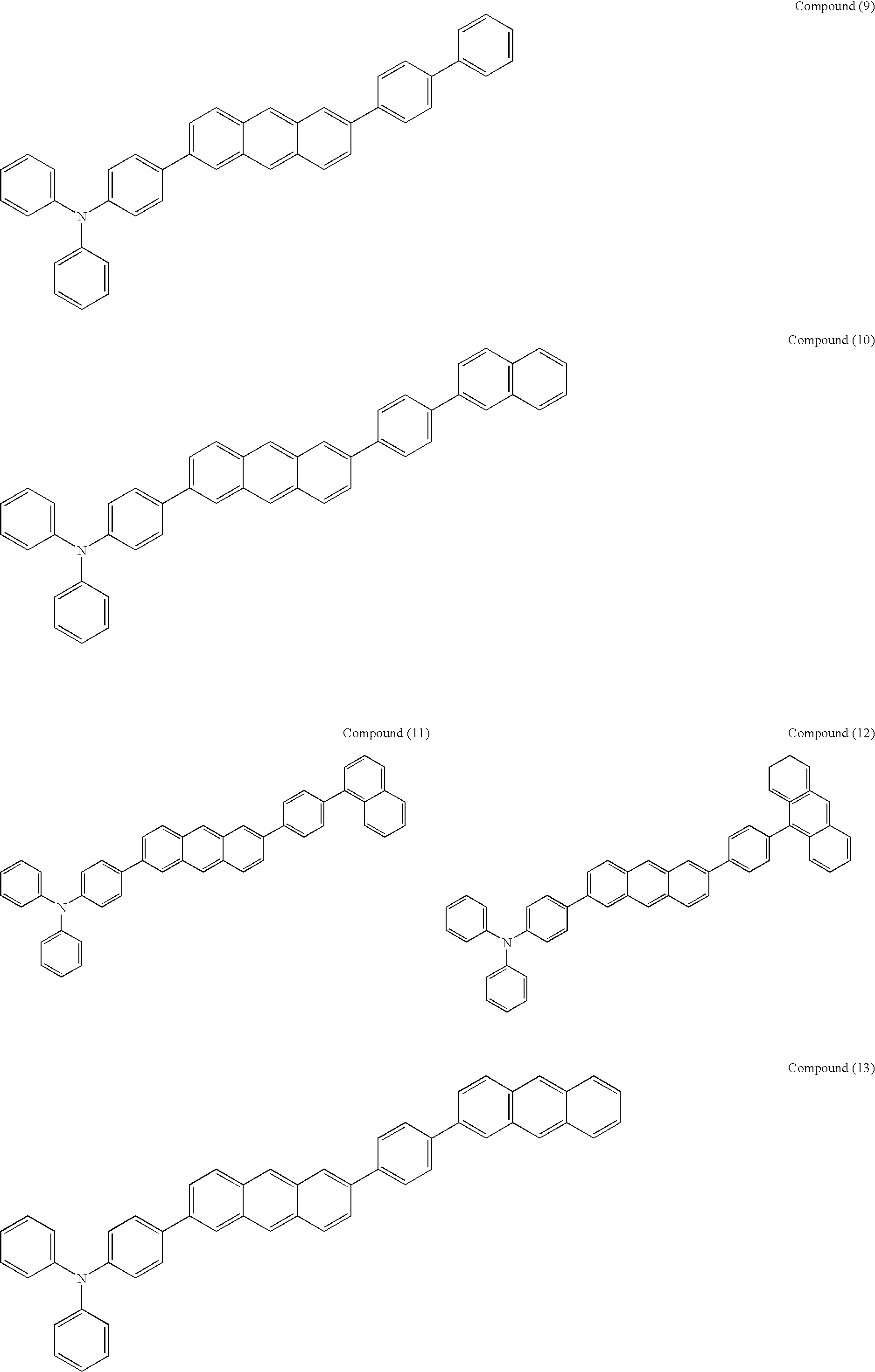

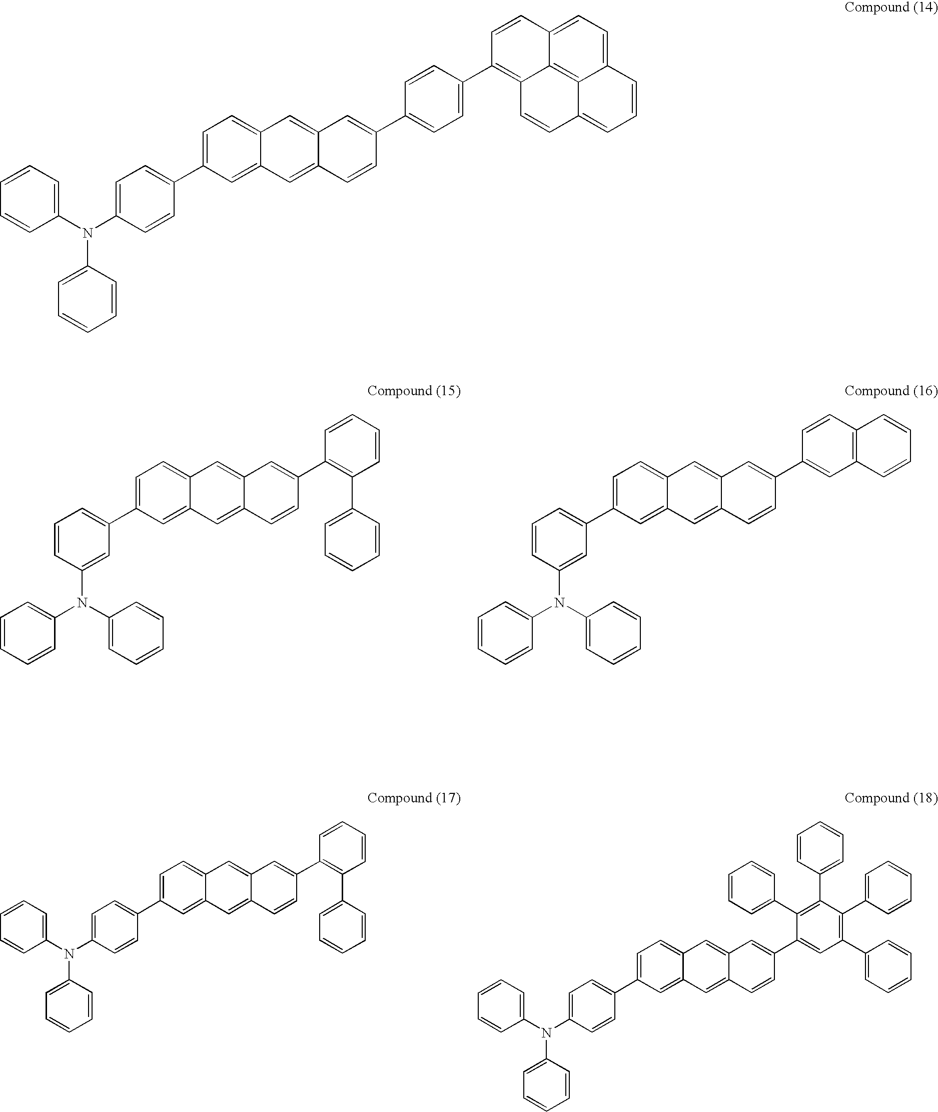

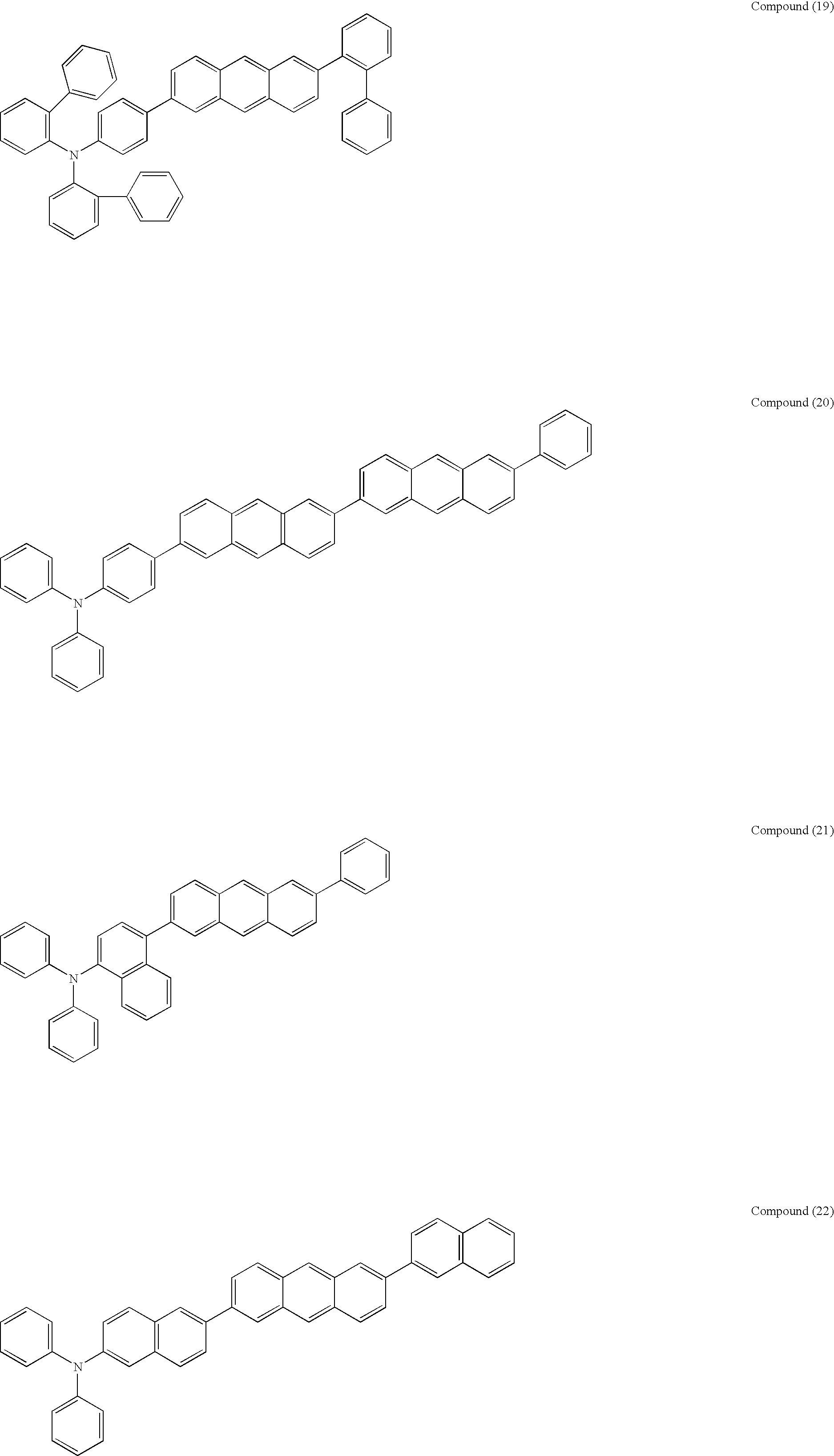

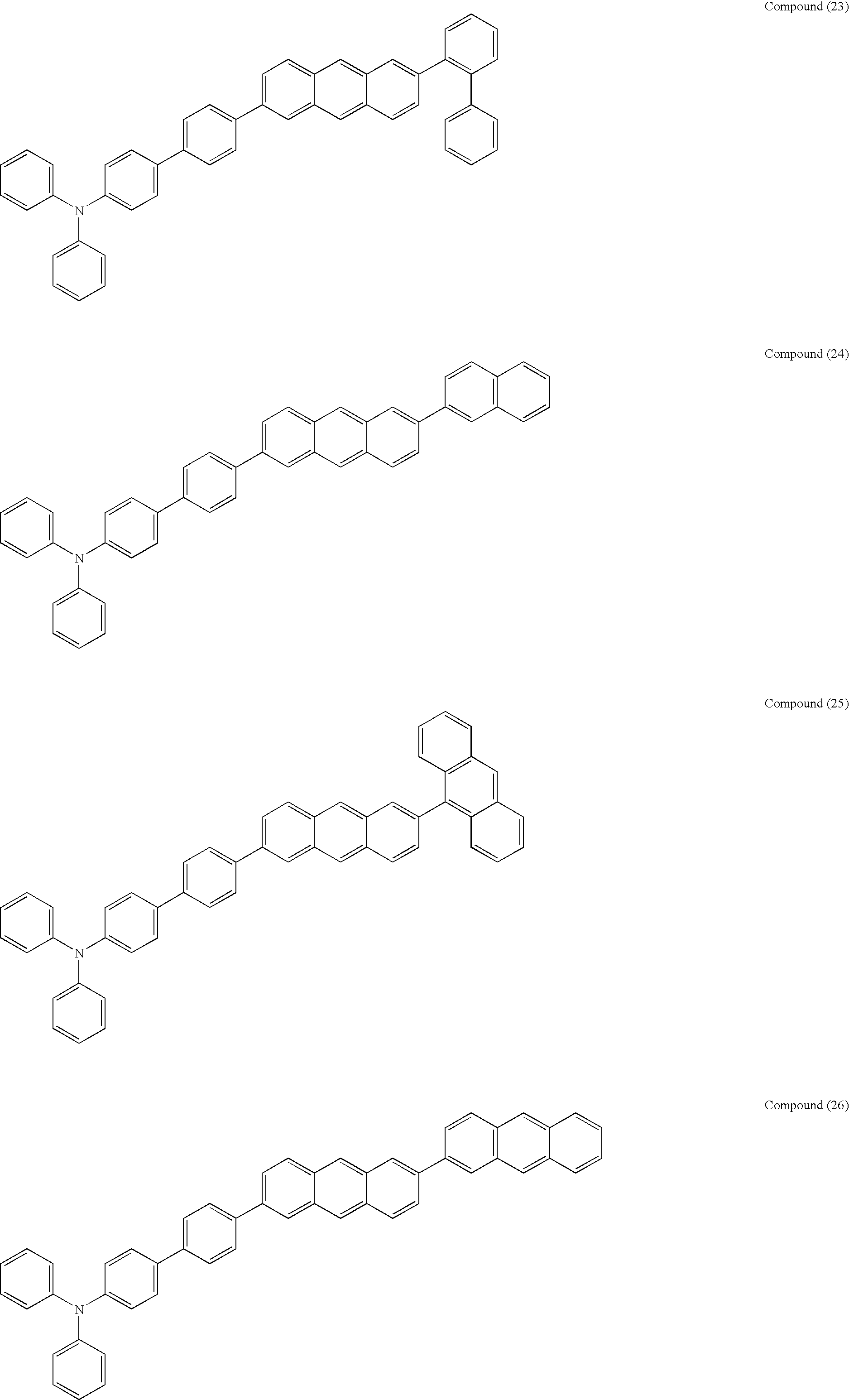

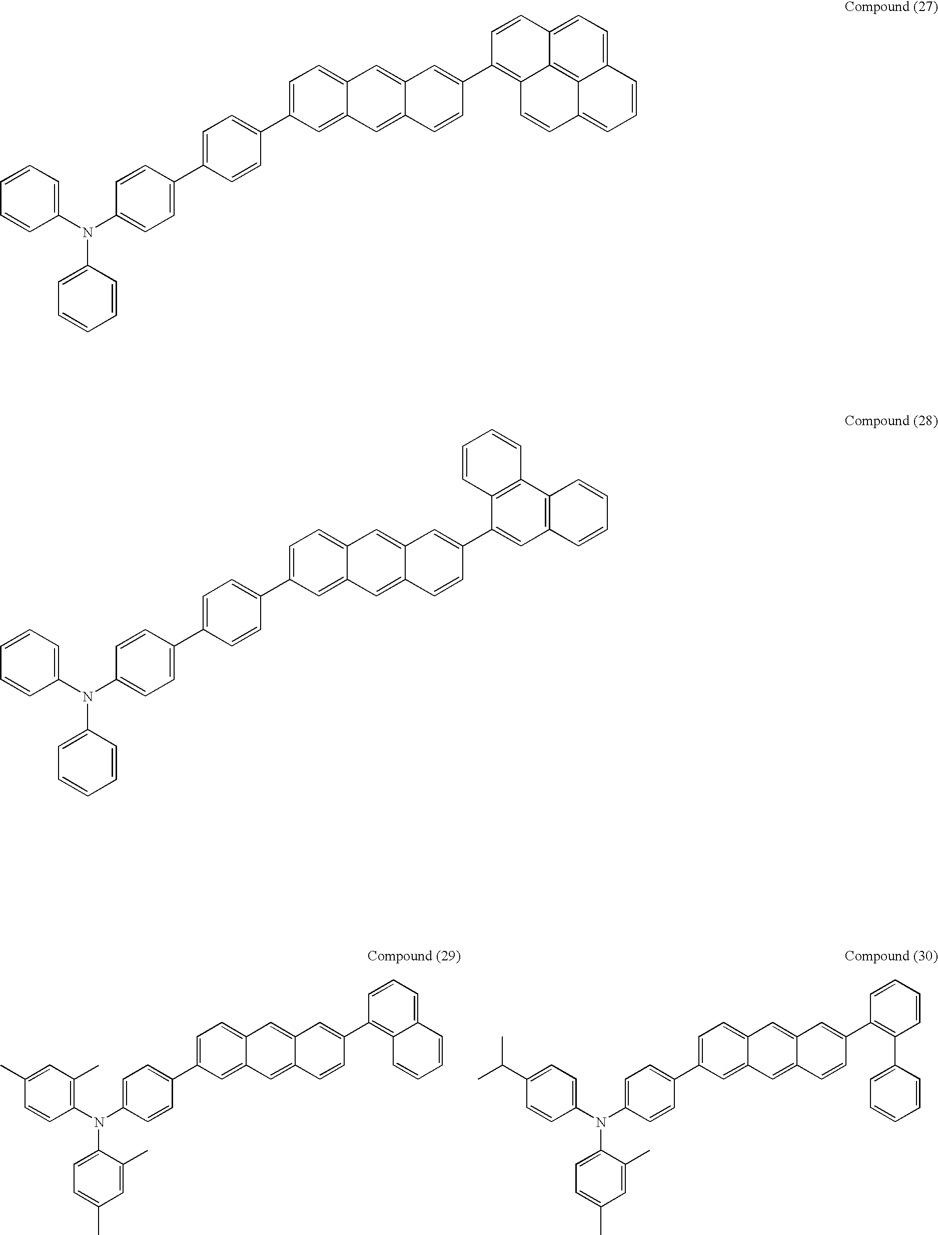

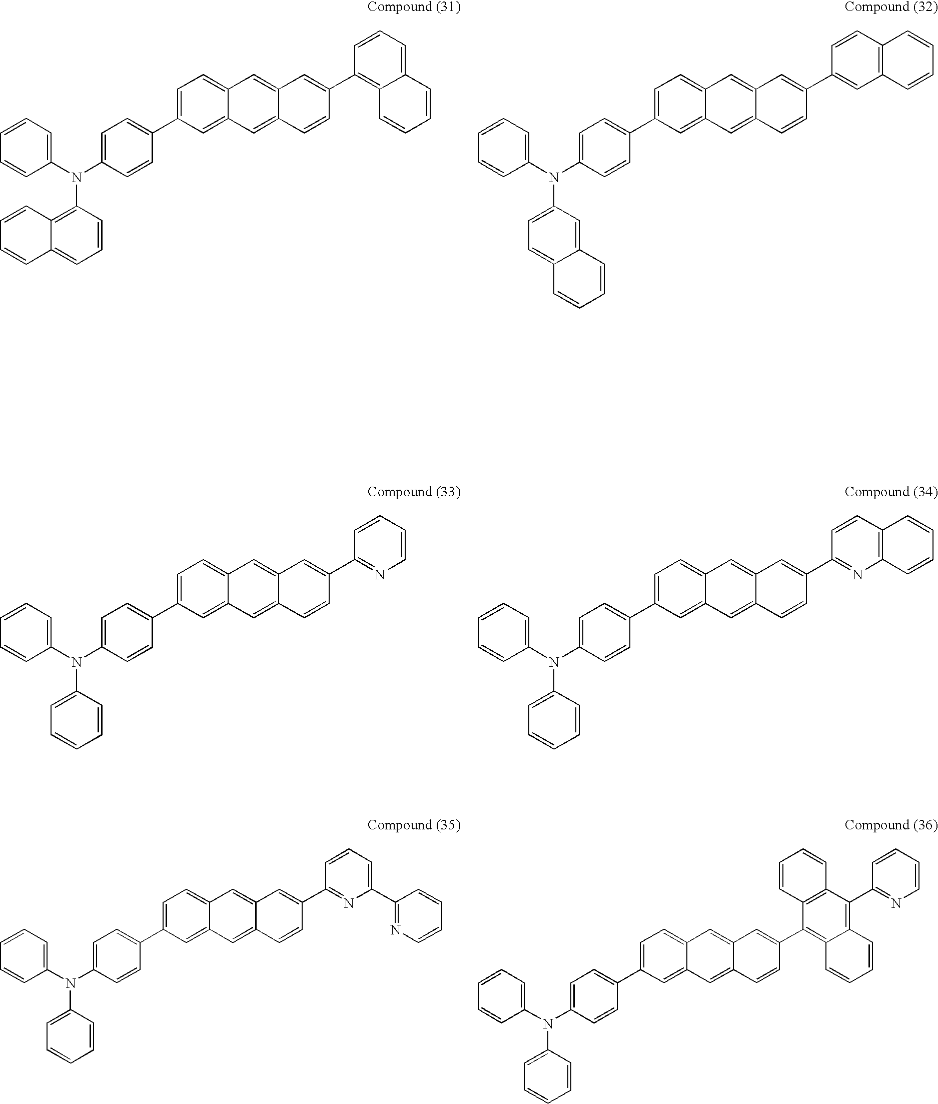

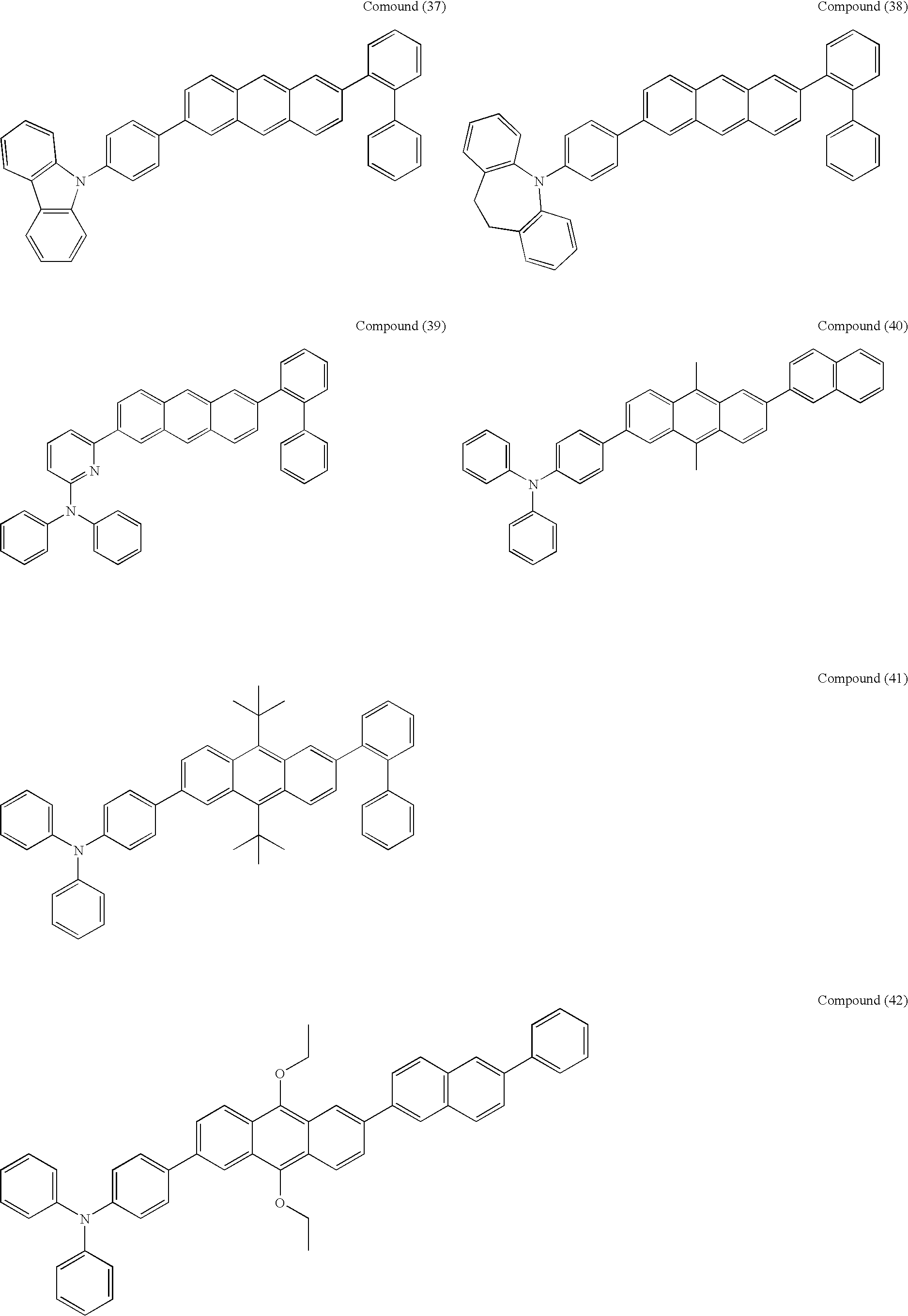

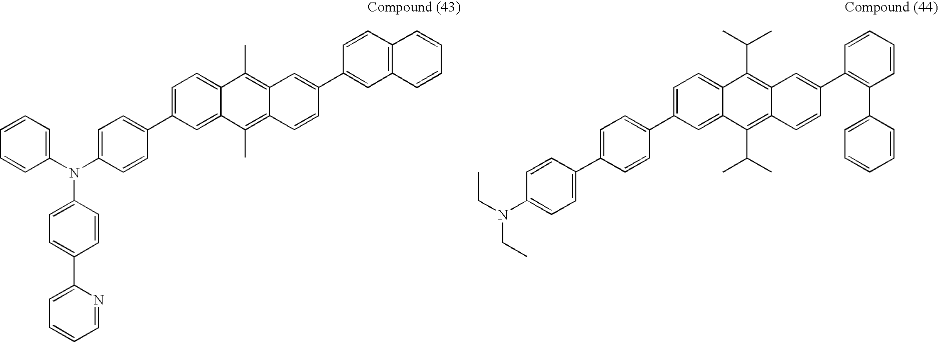

Compounds (45) to (96) will be shown below as the specific examples of the anthracene derivative represented by general formula (2). However, it is to be understood that the present invention is not limited thereto.

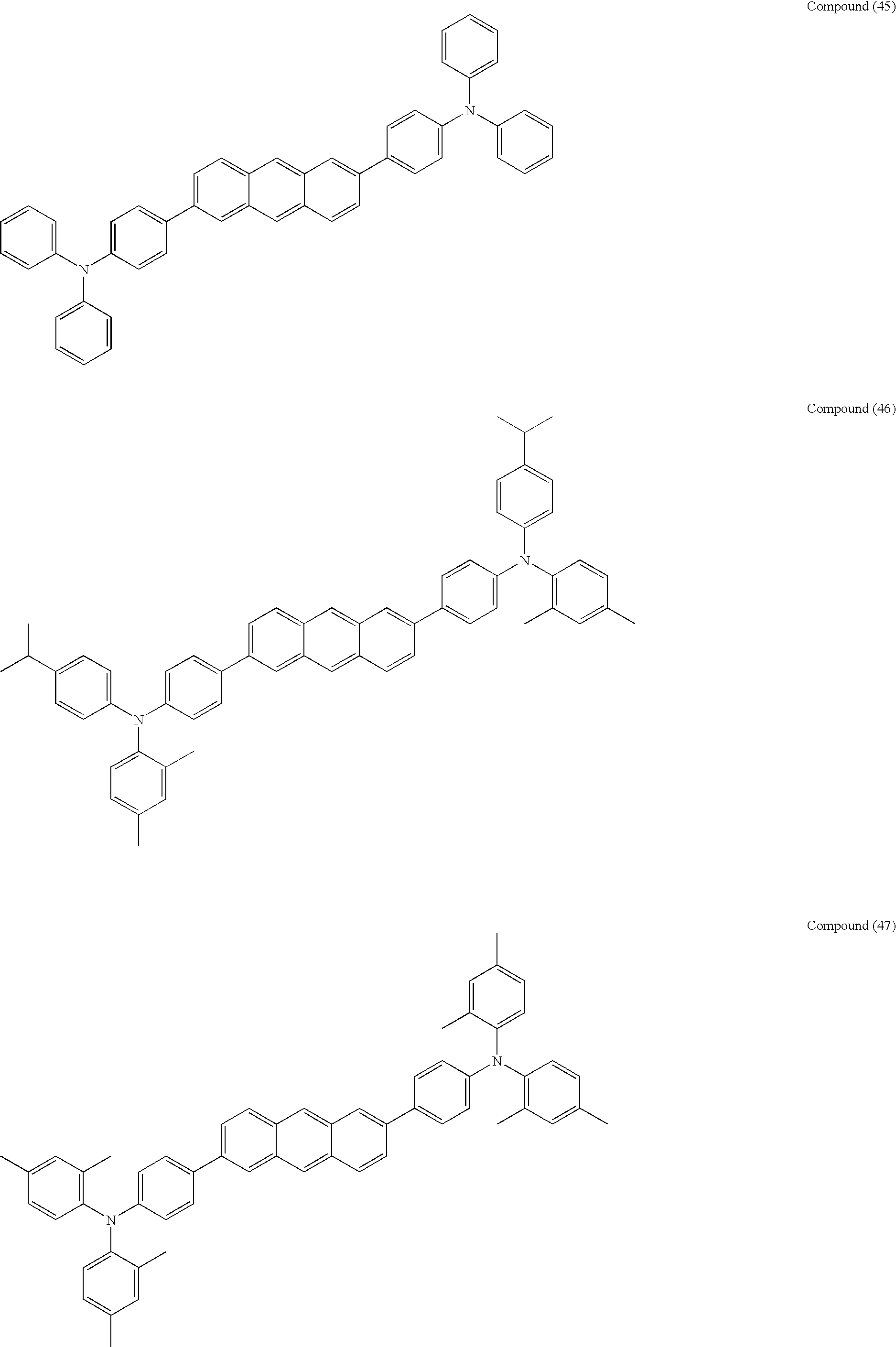

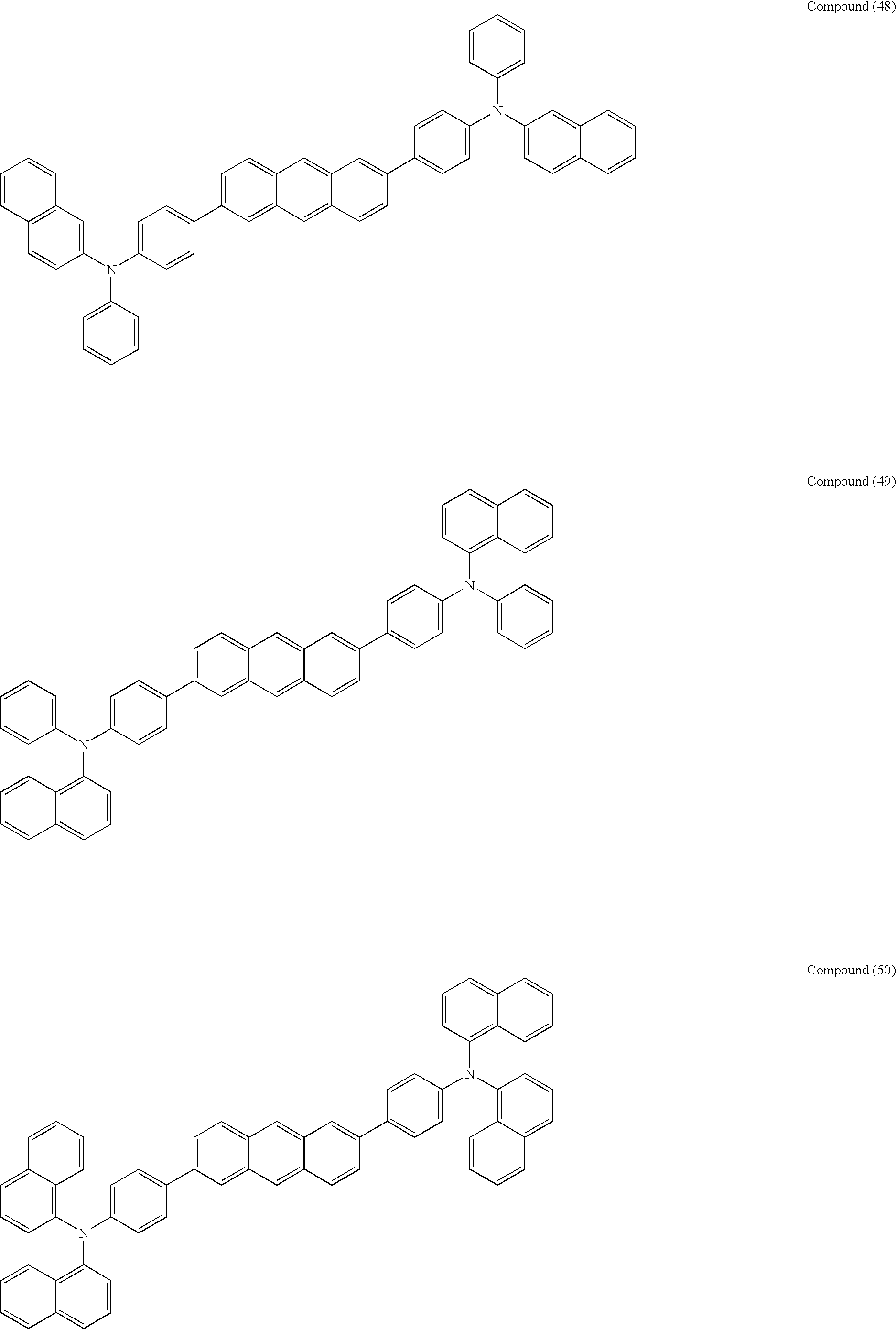

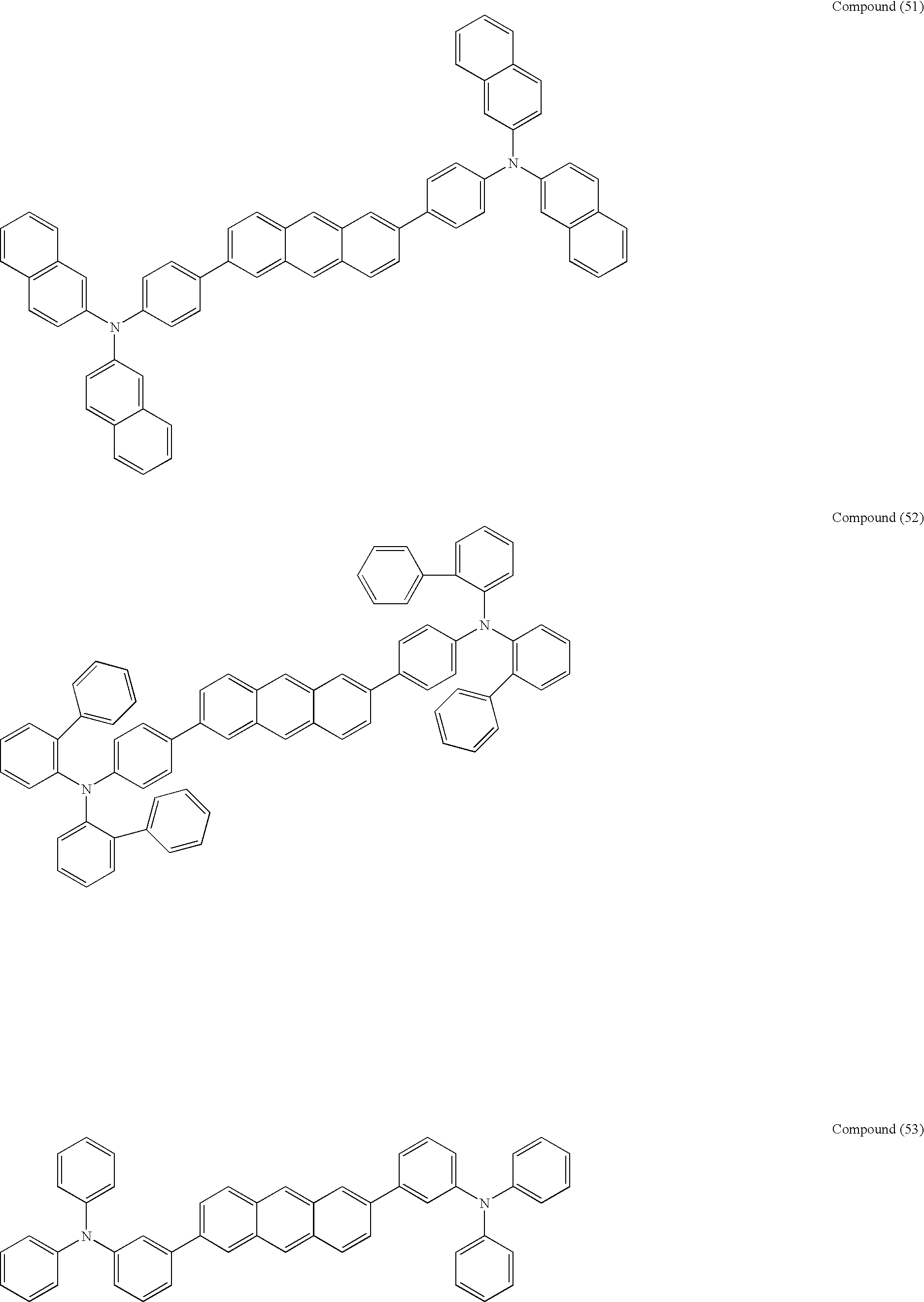

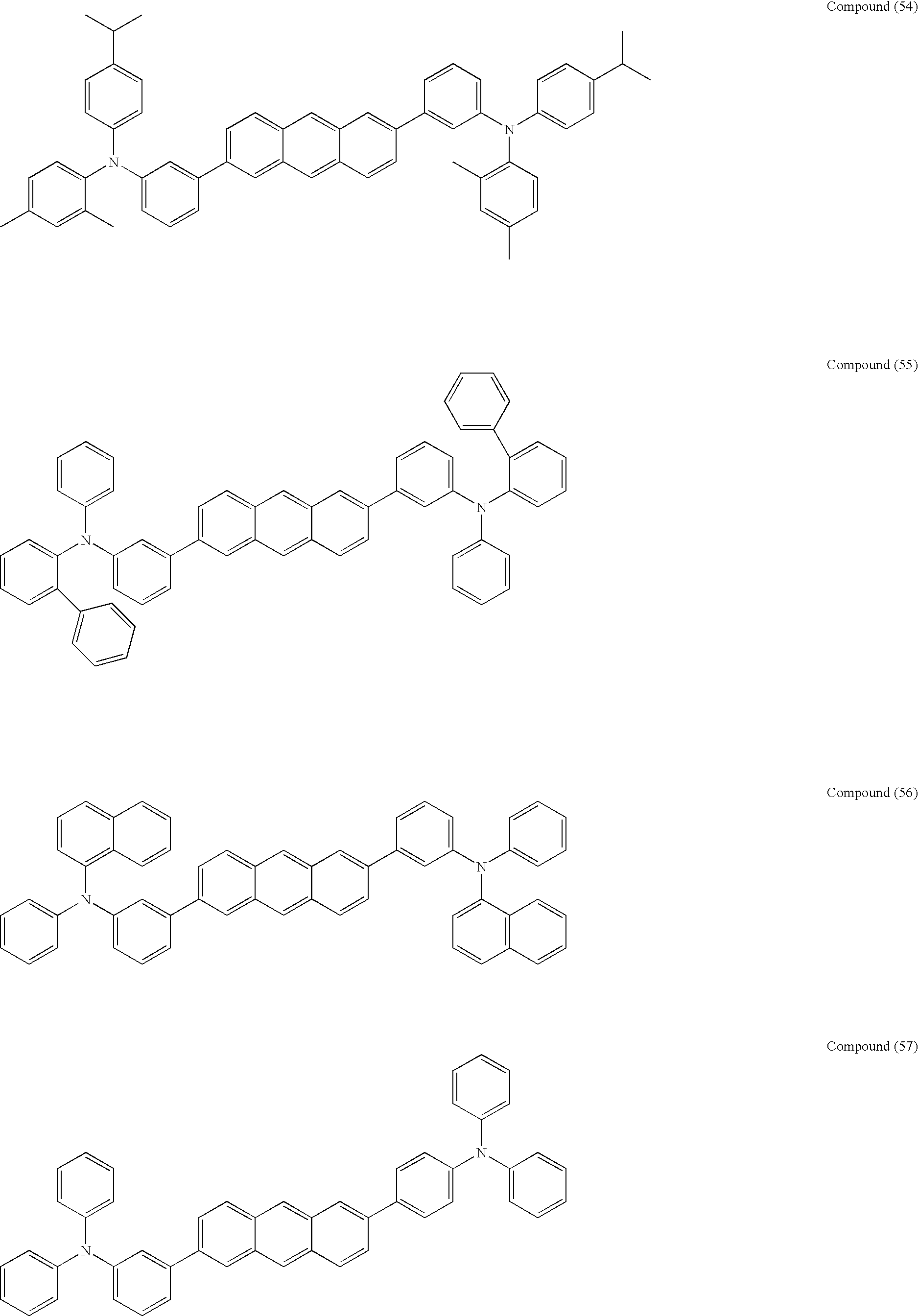

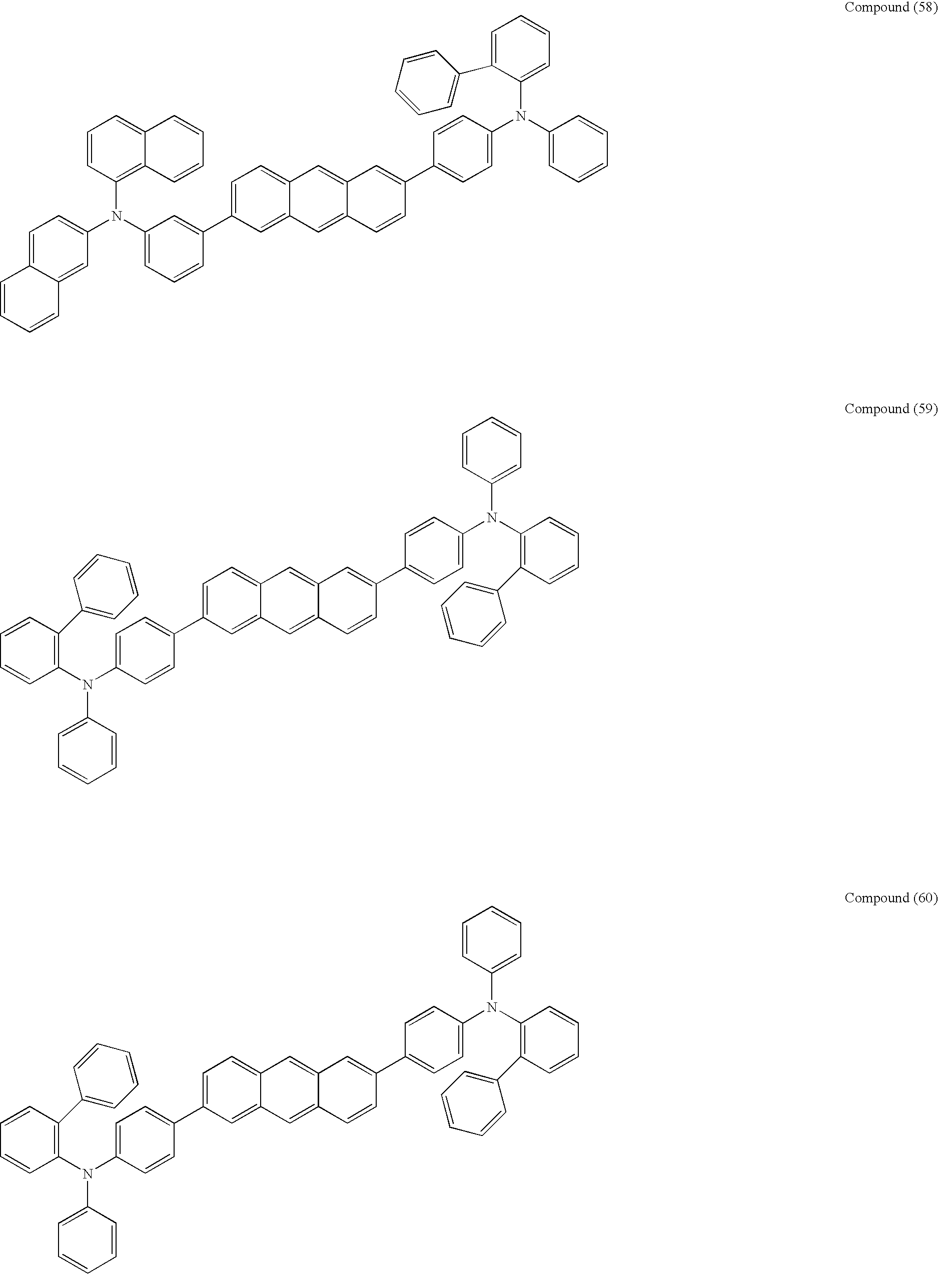

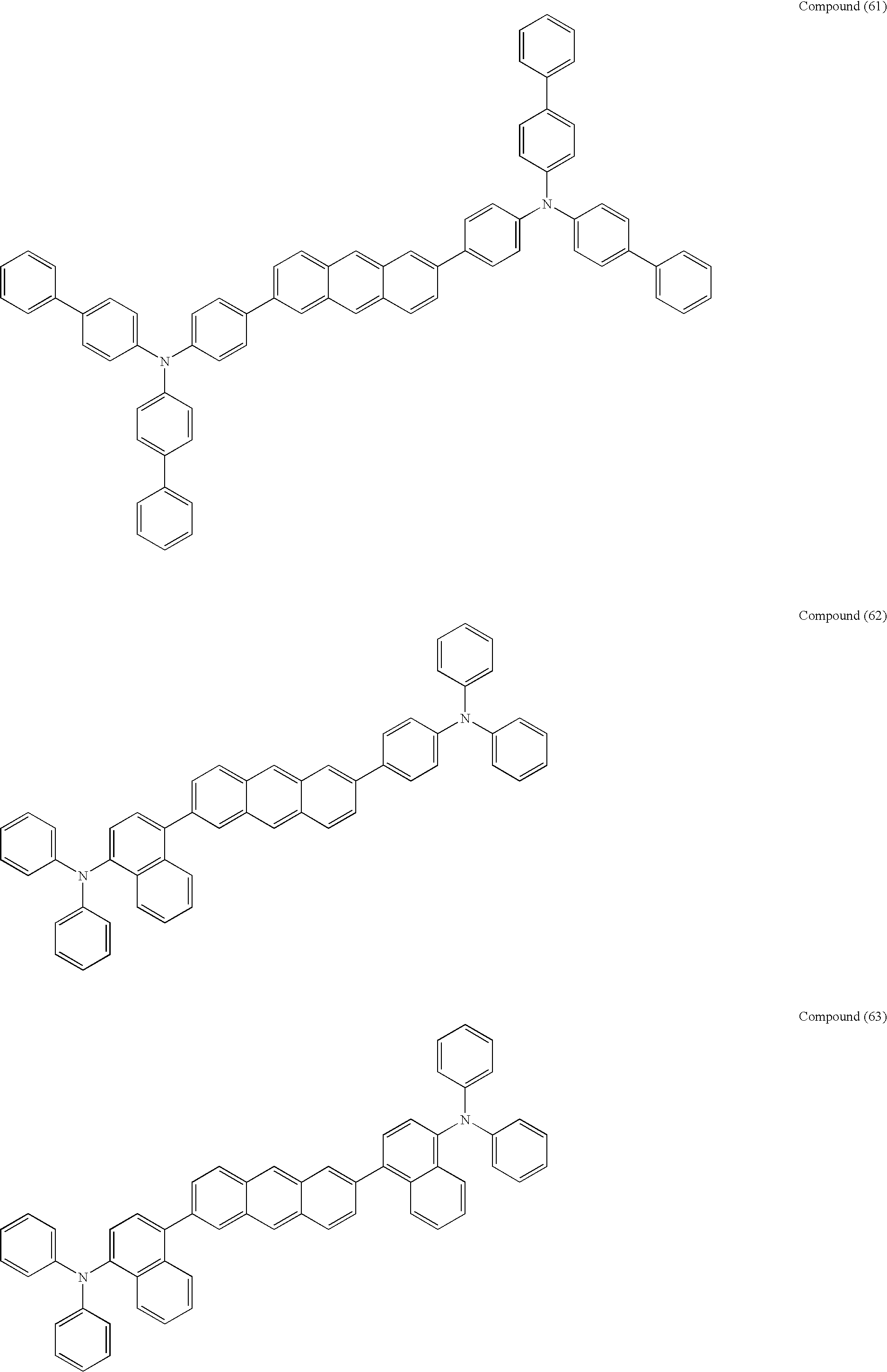

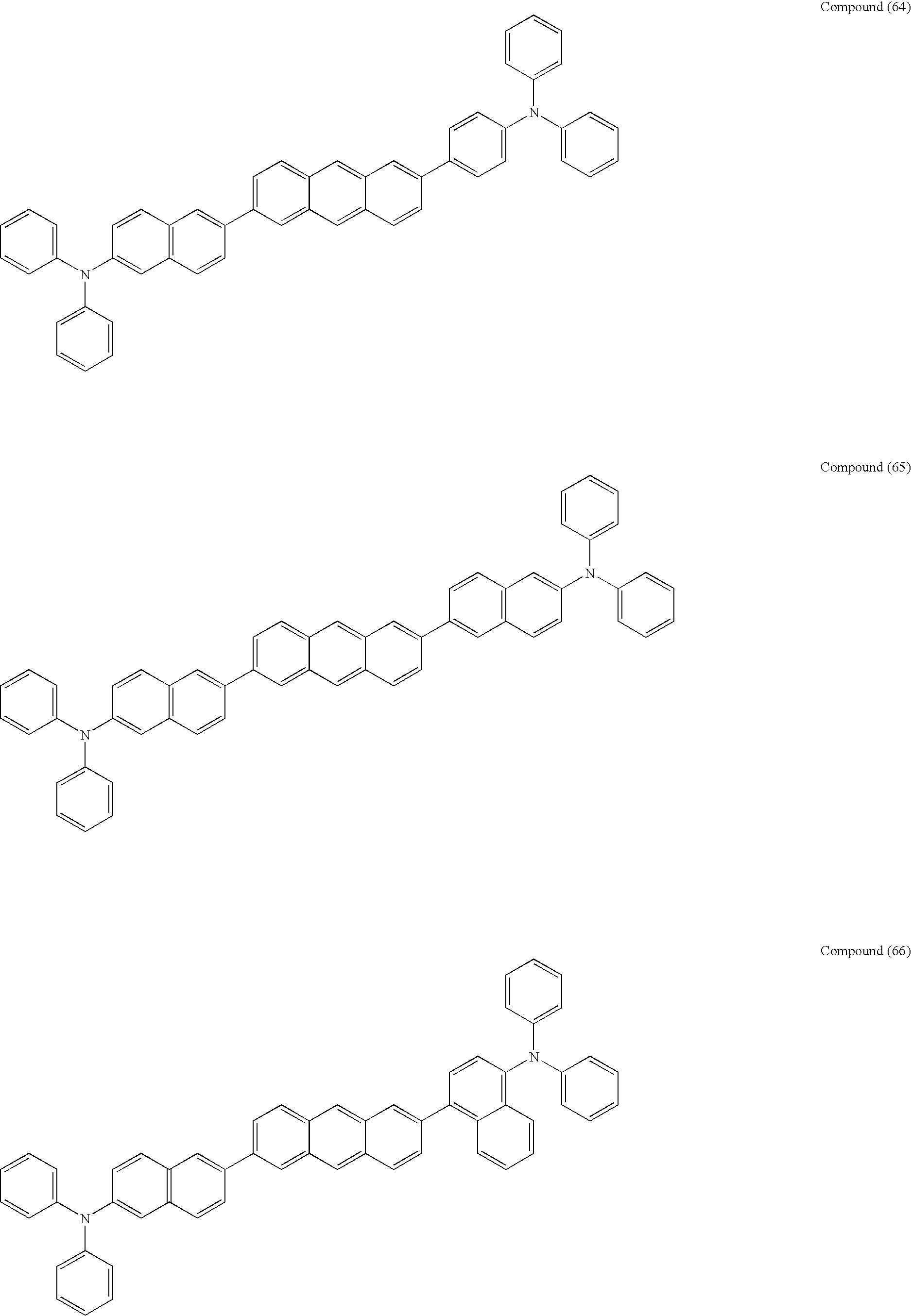

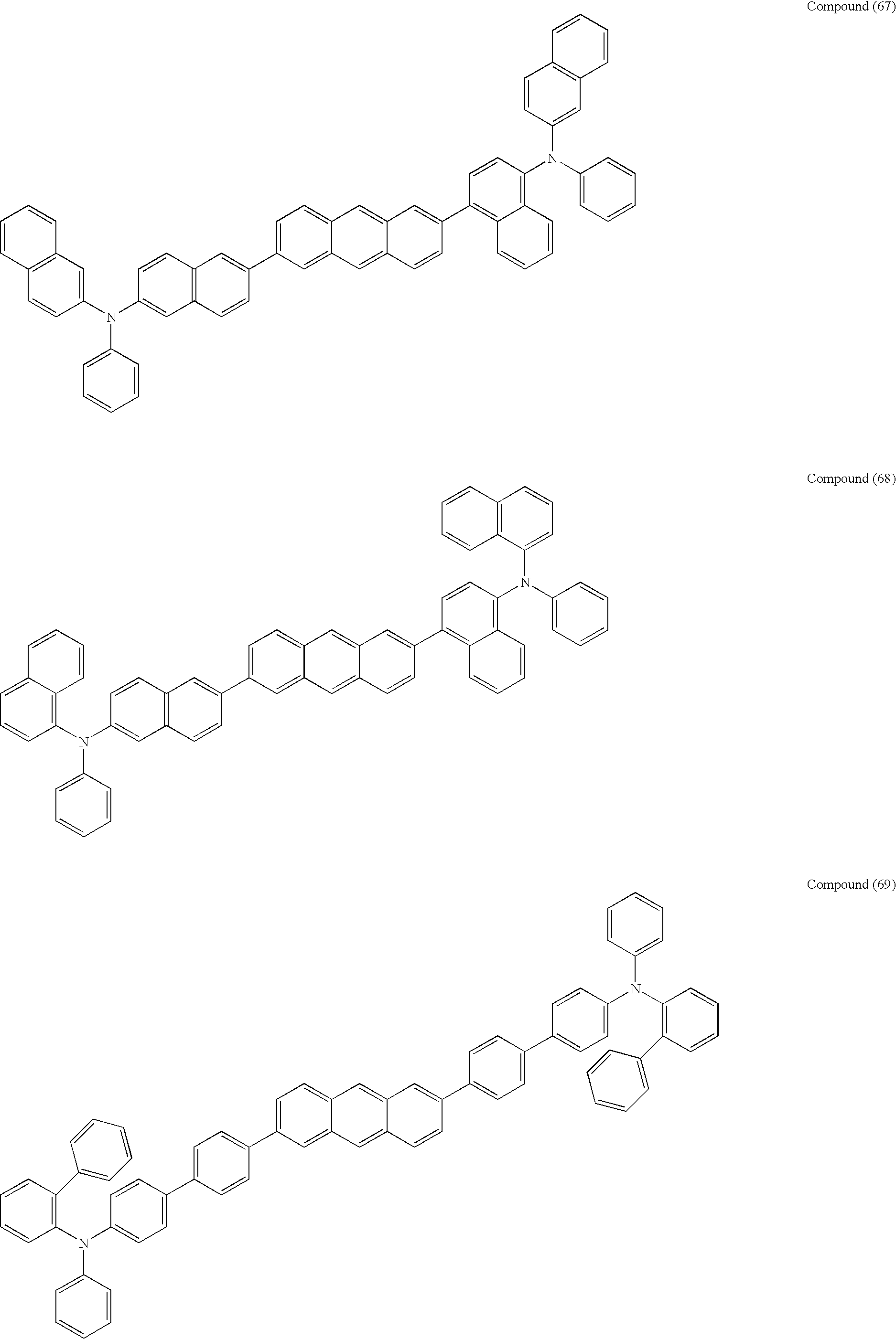

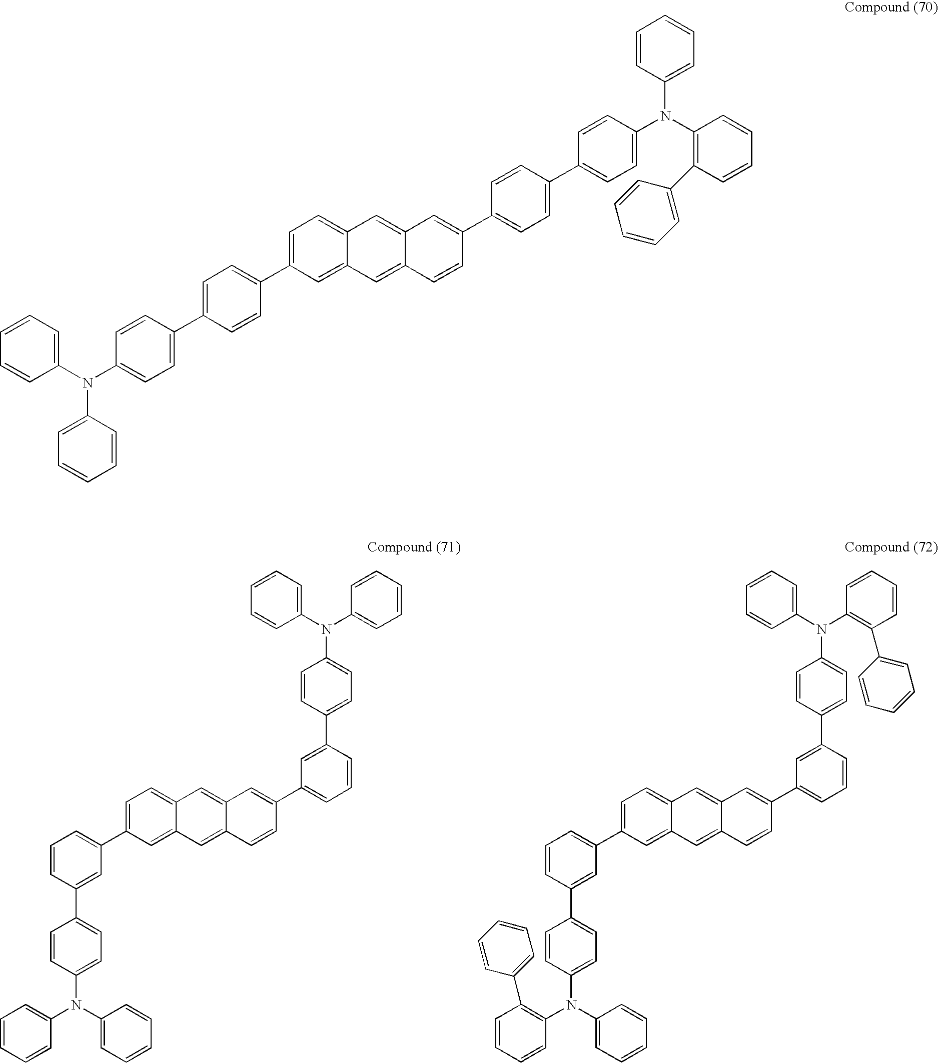

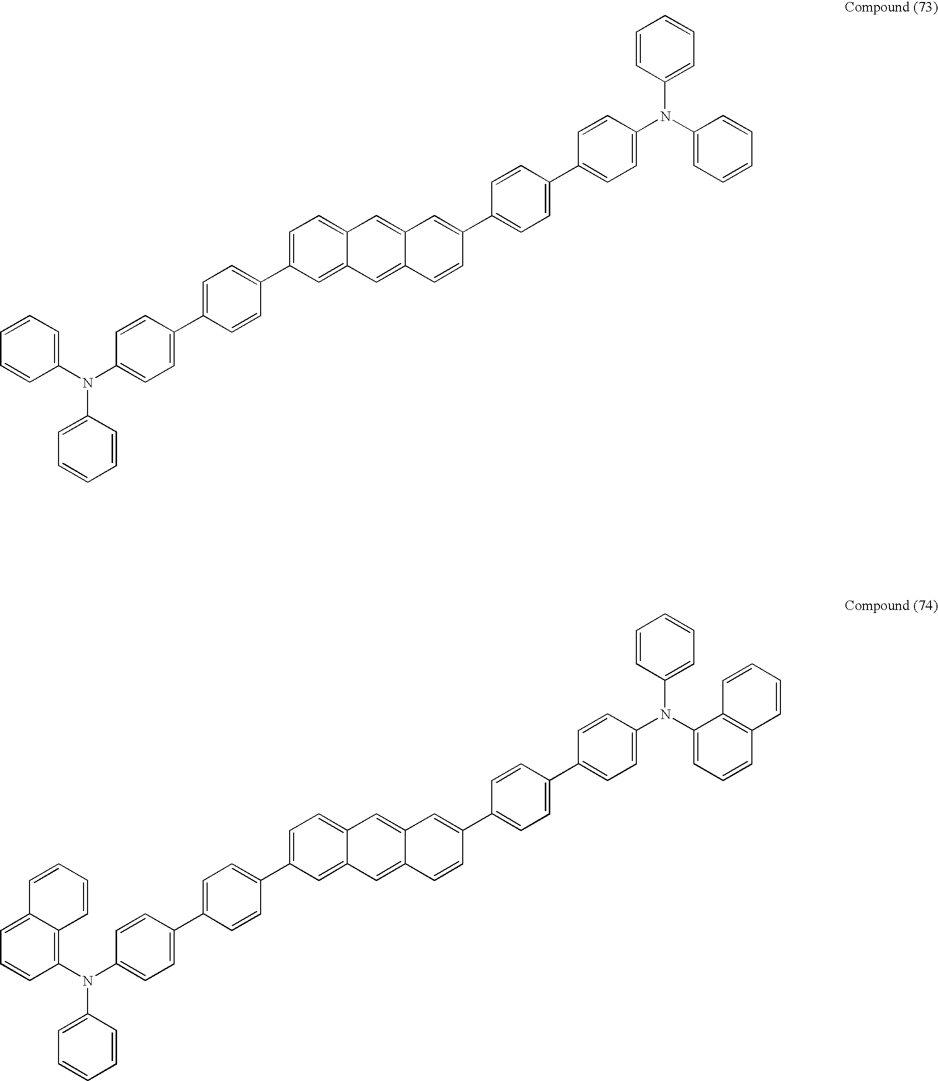

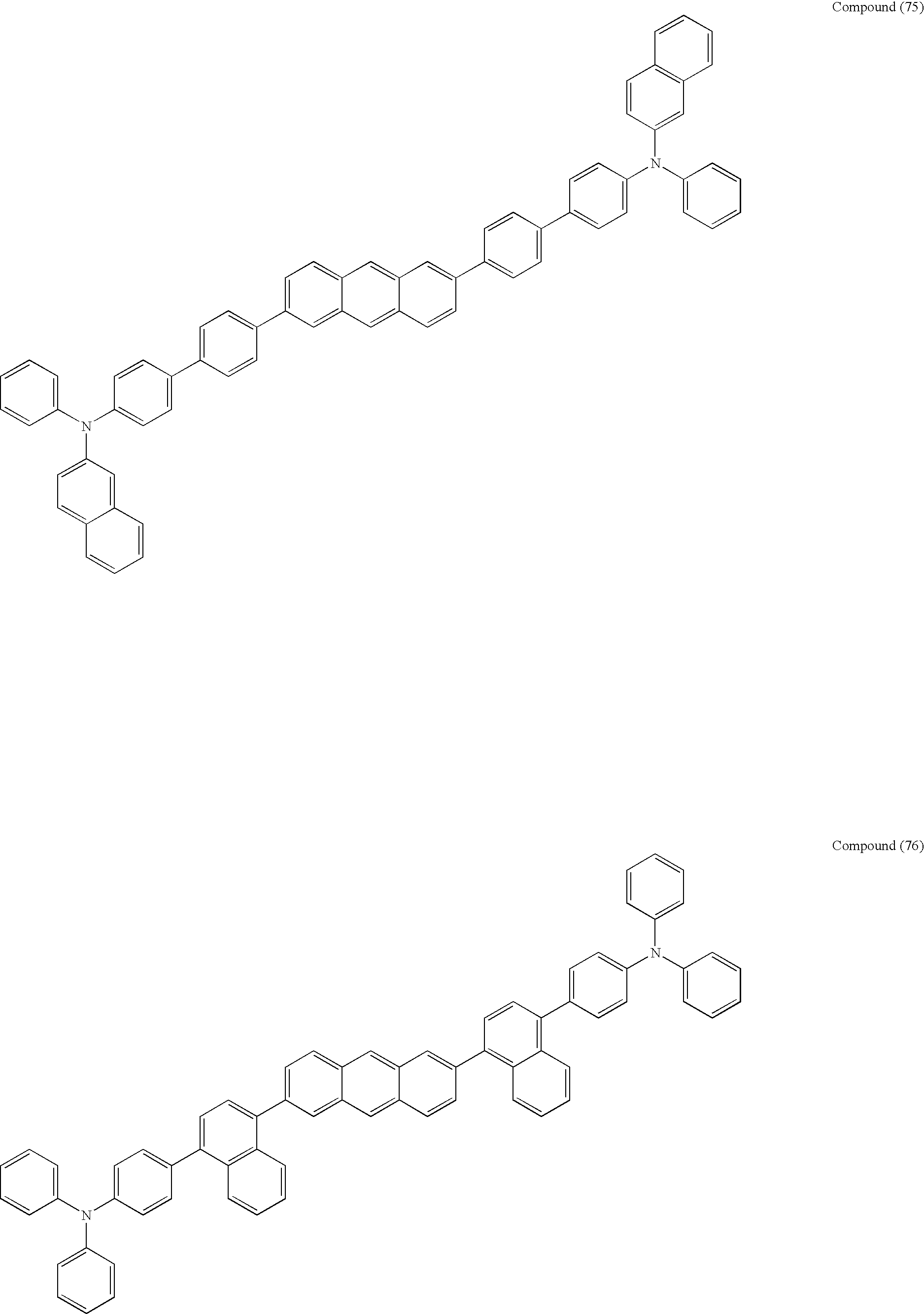

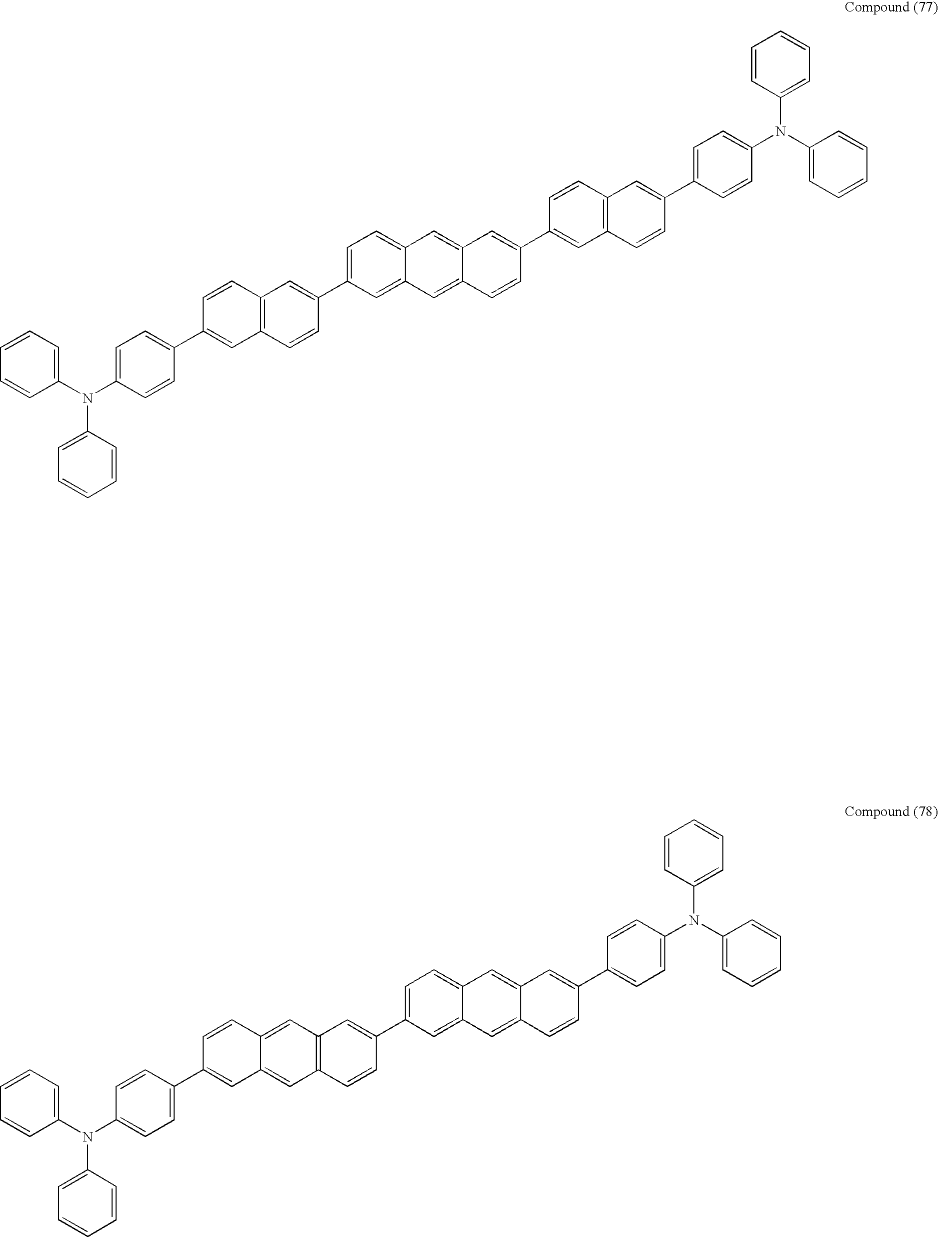

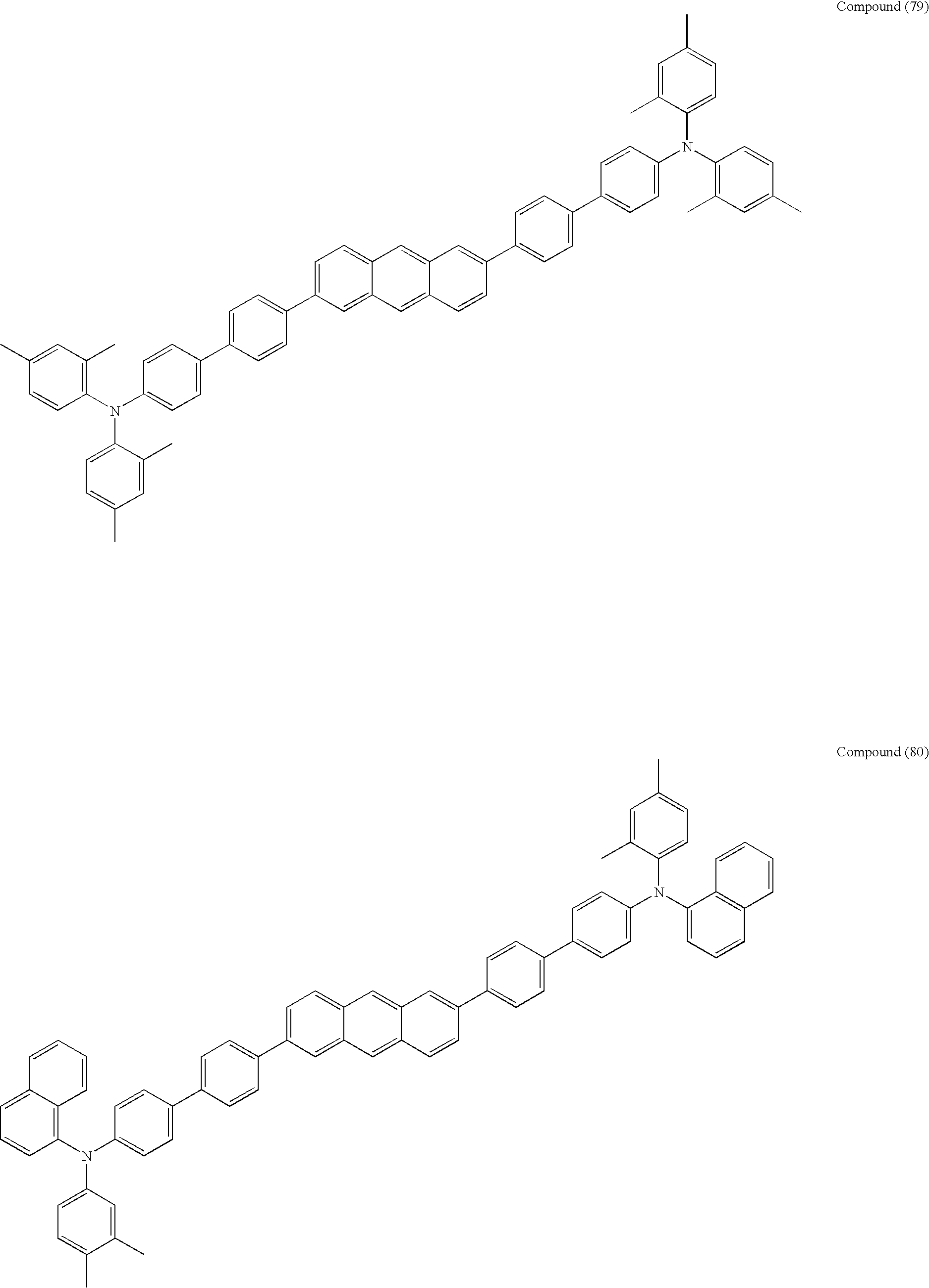

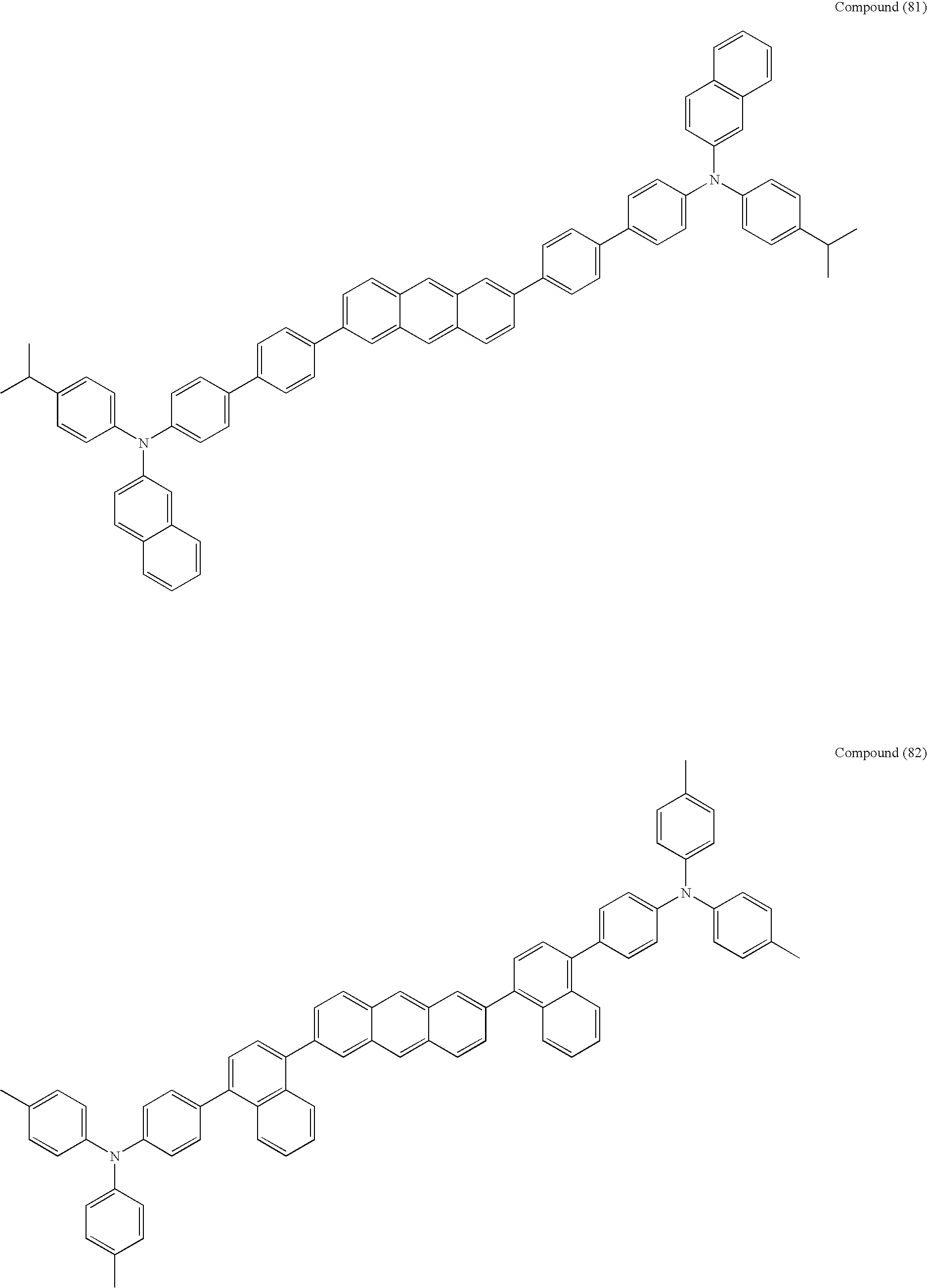

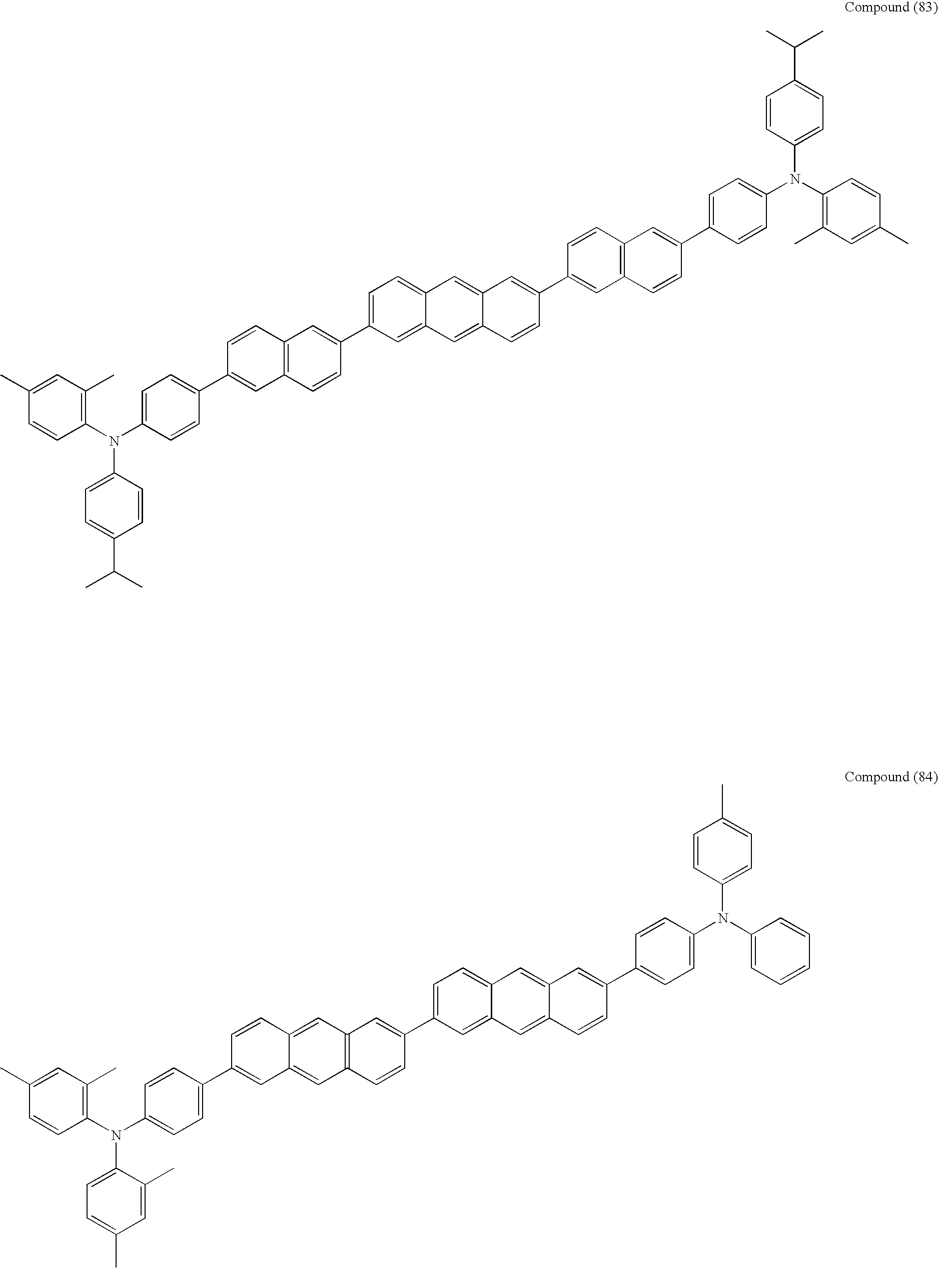

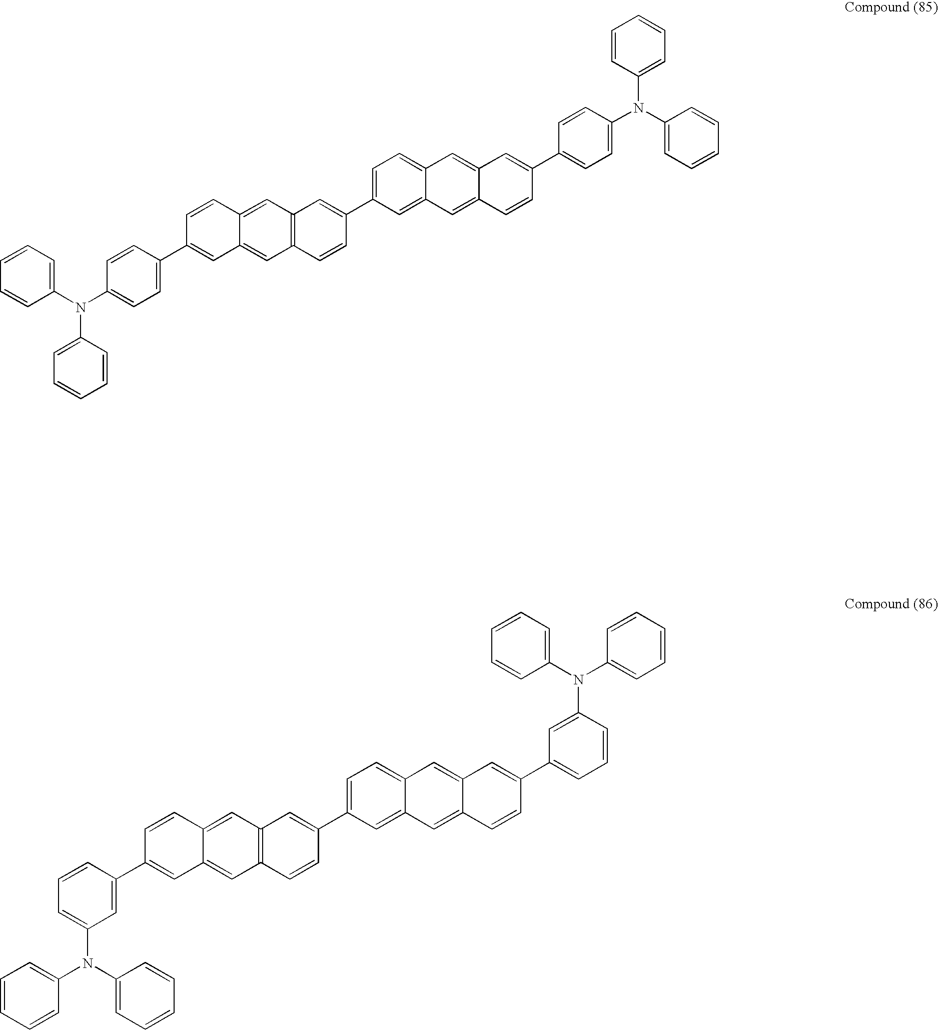

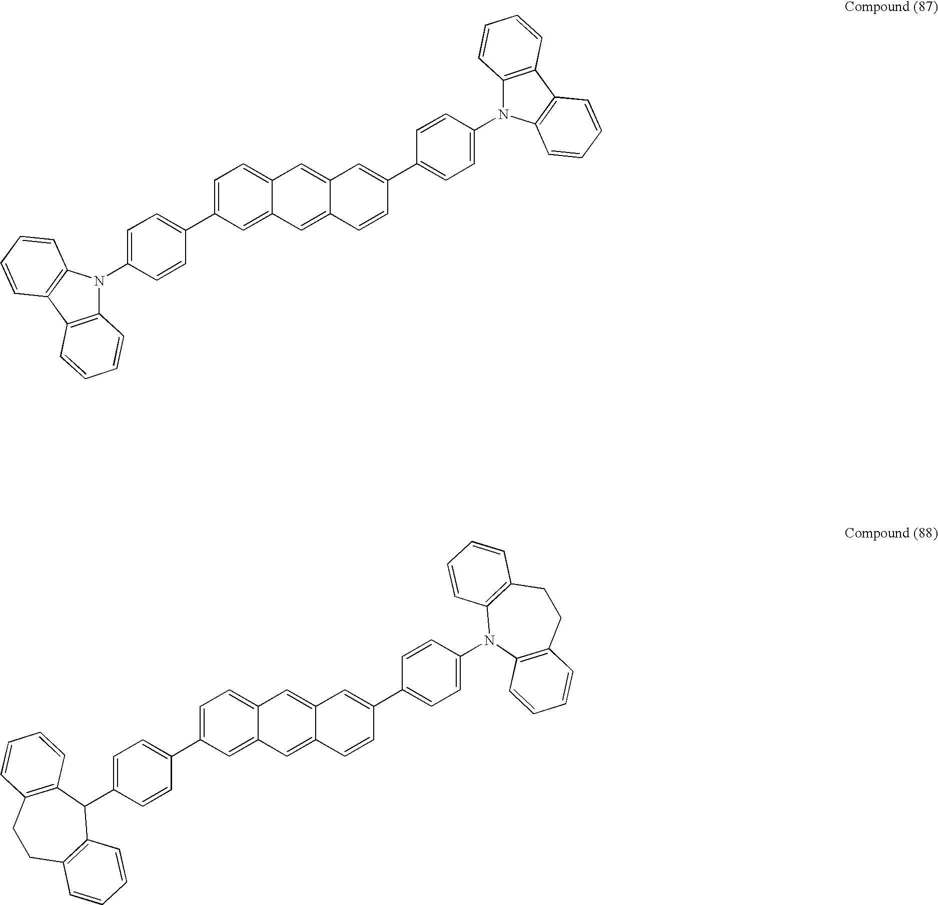

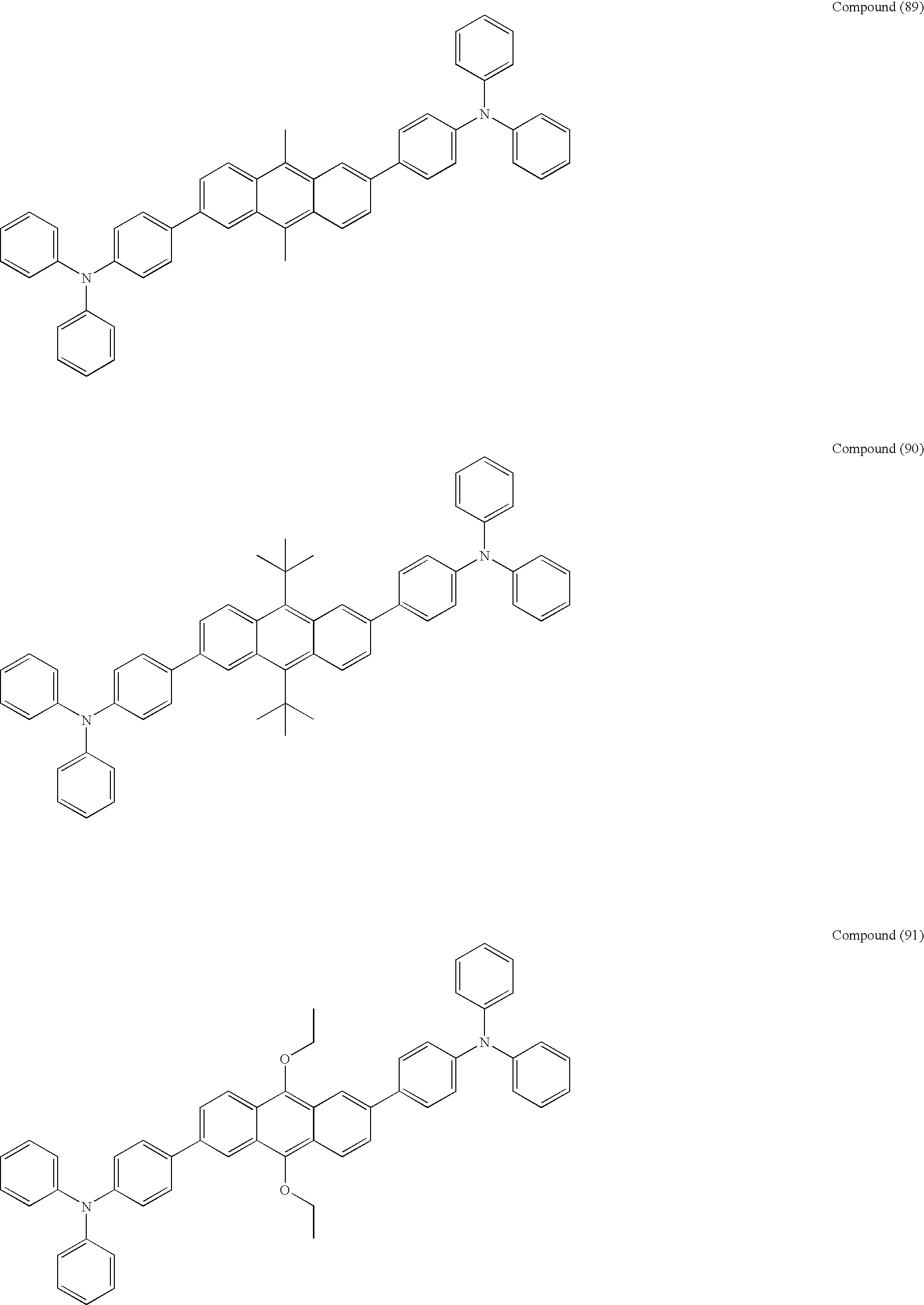

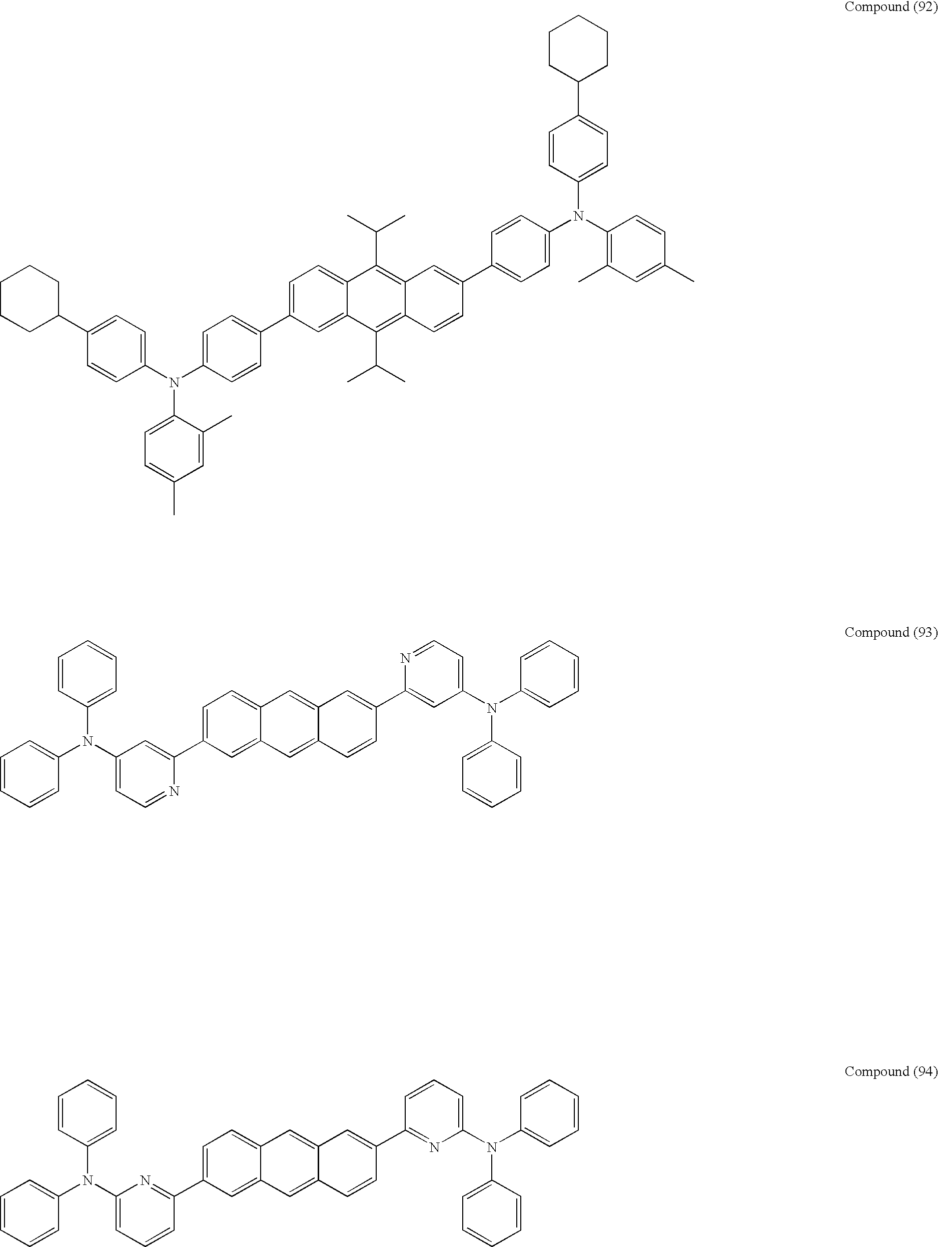

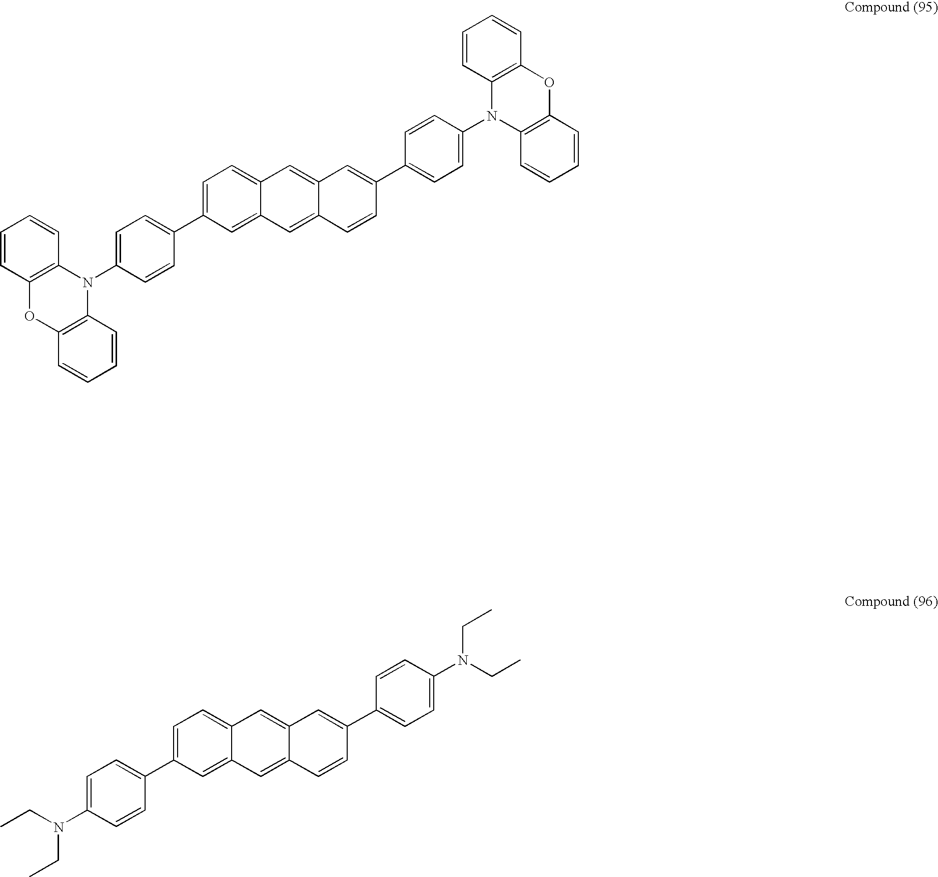

The anthracene derivative according to any of the embodiments of the present invention is used as a material constituting an organic layer of an organic electroluminescent device. Preferably, the purity of the anthracene derivative is increased before being used in a manufacturing process of an organic electroluminescent device. The purity is preferably 95% or more, and more preferably 99% or more. In order to obtain an organic compound with such a high purity, as the purification method after synthesis of the organic compound, recrystallization, reprecipitation, or column purification using silica or alumina may be used. In addition, a known method of increasing purity by sublimation purification may also be used. Furthermore, by repeating any of these purification methods or by combining different purification methods, it is possible to decrease the amount of mixtures, such as unreacted substances, reaction by-products, catalyst residues, or remaining solvents, in the organic luminescent material according to any of the embodiments of the present invention, and thus an organic electroluminescent device having more excellent characteristics can be obtained.

Organic Electroluminescent Device and Display Unit Including the Same

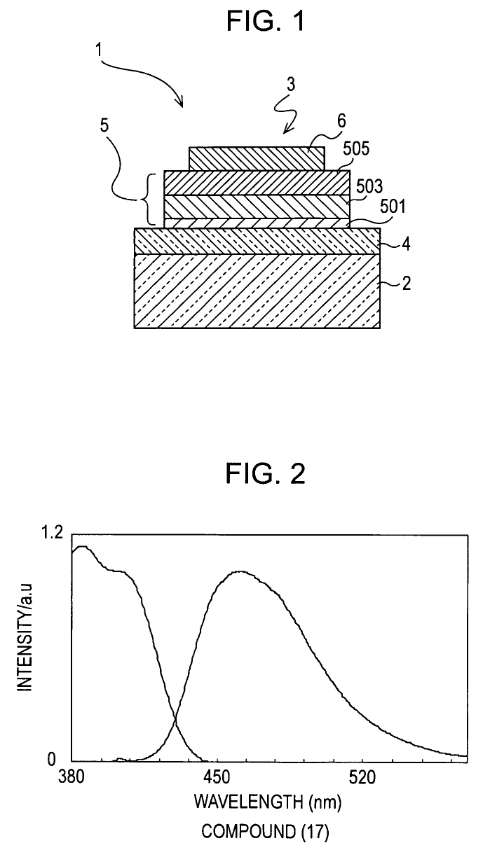

A structure of an organic electroluminescent device using the anthracene derivative described above and a display unit including the organic electroluminescent device will now be described in detail with reference to the drawing. FIG. 1 is a sectional view which schematically shows an organic electroluminescent device according to an embodiment of the present invention and a display unit including the organic electroluminescent device.

A display unit 1 shown in FIG. 1 includes a substrate 2 and an organic electroluminescent device 3 disposed on the substrate 2. The organic electroluminescent device 3 includes a lower electrode 4, an organic layer 5, and an upper electrode 6 laminated in that order on the substrate 2. Luminescence is extracted from the substrate 2 side or from the upper electrode 6 side. Although FIG. 1 shows a structure in which the organic electroluminescent device 3 for one pixel is disposed on the substrate 2, the display unit 1 is provided with a plurality of pixels, and a plurality of organic electroluminescent devices 3 are arrayed for the respective pixels.

Detailed structures of the individual components constituting the display unit 1, i.e., the substrate 2, the lower electrode 4, and the upper electrode 6 in that order, will now be described below.

The substrate 2 is made of glass, silicon, a plastic substrate, or a TFT substrate in which thin film transistors (TFTs) are disposed. In particular, when the display unit 1 is of a transmission type in which luminescence is extracted from the substrate 2 side, the substrate 2 is composed of a material having light transmission properties.

The lower electrode 4 disposed on the substrate 2 is used as an anode or a cathode. In the drawing, the case where the lower electrode 4 is an anode is shown as a typical example.

The lower electrode 4 is patterned into a shape suitable for the driving system of the display unit 1. For example, when the driving system of the display unit 1 is of a passive matrix type, the lower electrode 4 is, for example, formed into a stripe shape. When the driving system of the display unit 1 is of an active matrix type in which each pixel is provided with a TFT, the lower electrode 4 is patterned in accordance with the individual pixels arrayed and is formed so as to be connected to each TFT provided per pixel through a contact hole (not shown) formed in an interlayer insulating film covering the TFTs.

On the other hand, the upper electrode 6 disposed on the lower electrode 4 with the organic layer 5 therebetween is used as a cathode when the lower electrode 4 is an anode, and is used as an anode when the lower electrode 4 is a cathode. In the drawing, the case where the upper electrode 6 is a cathode is shown.

When the display unit 1 is of a passive matrix type, the upper electrode 6 is, for example, formed into a stripe shape intersecting with the stripe of the lower electrode 4, and laminated portions in which these stripes are intersecting with each other correspond to organic electroluminescent devices 3. When the display unit 1 is of an active matrix type, the upper electrode 6 is formed in the shape of a solid film so as to cover one face of the substrate 2, and is used as a common electrode for the individual pixels. When the driving system of the display unit 1 is of an active matrix type, in order to improve the open area ratio of the organic electroluminescent device 3, a top emission type in which luminescence is extracted from the upper electrode 6 side is preferably adopted.

As the anode material constituting the lower electrode 4 (or upper electrode 6), those having a work function as large as possible are desirable. Preferred examples thereof include nickel, silver, gold, platinum, palladium, selenium, rhodium, ruthenium, iridium, rhenium, tungsten, molybdenum, chromium, tantalum, niobium, alloys and oxides of these, tin oxide, indium tin oxide (ITO), zinc oxide, and titanium oxide.

On the other hand, as the cathode material constituting the upper electrode 6 (or lower electrode 4), those having a work function as small as possible are desirable. Preferred examples thereof include magnesium, calcium, indium, lithium, aluminum, silver, and alloys of these.

With respect to the electrode at the side from which luminescence generated in the organic electroluminescent device 3 is extracted, a material having light transmission properties is appropriately selected for use from the materials described above. In particular, a material that transmits 30% or more of light in the wavelength range of light emitted by the organic electroluminescent device 3 is preferably used.

For example, when the display unit 1 is of a transmission type in which luminescence is extracted from the substrate 2 side, an anode material having light transmission properties, such as ITO, is used for the lower electrode 4 serving as the anode, and a cathode material with good reflectance, such as aluminum, is used for the upper electrode 6 serving as the cathode.

On the other hand, when the display unit 1 is of a top emission type in which luminescence is extracted from the upper electrode 6 side, an anode material, such as chromium or a silver alloy, is used for the lower electrode 4 serving as an anode, and a cathode material having light transmission properties, such as a compound of magnesium and silver (MgAg), is used for the upper electrode 6 serving as a cathode. However, since MgAg has a light transmittance of about 30% in the wavelength range of green, the organic layer 5, which will be described below, is preferably designed such that a resonator structure is optimized to increase the intensity of light that is extracted.

The organic layer 5 sandwiched between the lower electrode 4 and the upper electrode 6 includes a hole-transport layer 501, a luminescent layer 503, and an electron-transport layer 505 laminated in that order on the anode (the lower electrode 4 in the drawing).

As the hole-transport layer 501, a known material, such as NPB [N,N′-bis(1-naphthyl)-N,N′-diphenyl(1,1′-biphenyl)-4,4′-diamine], triphenylamine dimer, trimer, or tetramer, or a starburst amine, can be used in the form of a single layer or a multi-layer film, or in combination as a mixture.

The luminescent layer 503 disposed on the hole-transport layer 501 is characteristic to the present invention and contains the anthracene derivative represented by general formula (1) or (2) with compounds (1) to (96) being mentioned as examples thereof. The anthracene derivative according to any of the embodiments of the present invention has a high hole-transport property. Consequently, if the anthracene derivative is used alone or in a high concentration of 50% by volume or more, or if the anthracene derivative is used in mixture with other materials having a hole-transport property, luminescence from the electron-transport layer 505, which will be described below, is observed, resulting in a decrease in luminous efficiency in the luminescent layer 503 itself. Therefore, in such a case, preferably, a hole block layer is provided between the luminescent layer 503 and the electron-transport layer 505.

More preferably, the anthracene derivative according to any of the embodiments of the present invention is introduced as a guest into the luminescent layer 503, and the concentration of the anthracene derivative in the luminescent layer 503 is desirably 1% by volume or more and less than 50% by volume, preferably 1% to 20% by volume, and more preferably 1% to 10% by volume.

As a host material which is mixed with the anthracene derivative for use, a known material, such as oxadiazole, triazole, benzimidazole, silole, styrylarylene, paraphenylene, spiro paraphenylene, or an arylanthracene derivative, can be used.

For the electron-transport layer 505 disposed on the luminescent layer 503 having such a structure, a known material, such as Alq3, oxadiazole, triazole, benzimidazole, or a silole derivative, can be used.

Additionally, although not shown in the drawing, a hole injection layer may be interposed between the lower electrode 4 serving as the anode and the hole-transport layer 501. As the hole injection layer, a known material, such as a conductive polymer, e.g., polyphenylene vinylene (PPV), copper phthalocyanine, a starburst amine, or triphenylamine dimer, trimer, or tetramer, can be used in the form of a single layer or a multi-layer film, or in combination as a mixture. By interposing such a hole injection layer, hole injection efficiency is improved, which is more preferable.

Furthermore, although not shown in the drawing, an electron injection layer may be interposed between the electron-transport layer 505 and the cathode (upper electrode) 6. The electron injection layer can be made of an alkali metal oxide, an alkali metal fluoride, an alkaline-earth oxide, or an alkaline-earth fluoride, such as lithium oxide, lithium fluoride, cesium iodide, or strontium fluoride. By interposing such an electron injection layer, electron injection efficiency is improved, which is more preferable.

The formation of the organic layer 5 having a layered structure including the materials described above can be performed using each organic material synthesized by a known process and using a known method, such as vacuum deposition or spin coating.

Although not shown in the drawing, with respect to the display unit 1 including the organic electroluminescent device 3 having such a structure, in order to prevent the organic electroluminescent device 3 from being degraded due to water, oxygen, etc., in the atmosphere, it is desirable to form a sealing film made of magnesium fluoride or silicon nitride (SiNx) on the substrate 2 so as to cover the organic electroluminescent device 3. Alternatively, desirably, the organic electroluminescent device 3 is covered with a sealing can, and a hollow portion is purged with a dried inert gas or evacuated.

Although not shown in the drawing, with respect to the display unit 1 including the organic electroluminescent device 3 having such a structure, the organic electroluminescent device 3 may be allowed to serve as a blue luminescent device, and a red luminescent device and a green luminescent device each are provided per pixel along with the blue luminescent device, these three pixels being allowed to serve as subpixels to constitute one pixel. A plurality of pixels each composed of a group of three subpixels may be arrayed on the substrate 2 to perform full-color display.

Examples in which the anthracene derivative according to any of the embodiments of the present invention is used for the luminescent layer 503 have been described above. However, since the anthracene derivative according to any of the embodiments of the present invention has a high hole-transport property, the anthracene derivative may be used as a material constituting the hole-transport layer 501 or the hole injection layer, and may be used as a doping material for these layers.

### EXAMPLES

Synthesis Examples 1 to 10 of anthracene derivatives according to the embodiments of the present invention and Examples 1 to 34 of organic electroluminescent devices using the anthracene derivatives according to the embodiments of the present invention will be described below.

### Synthesis Example 1

### Synthesis of Compound (2)

First, with reference to synthesis formula (1), 2,6-dibromoanthracene was synthesized by the process described below.

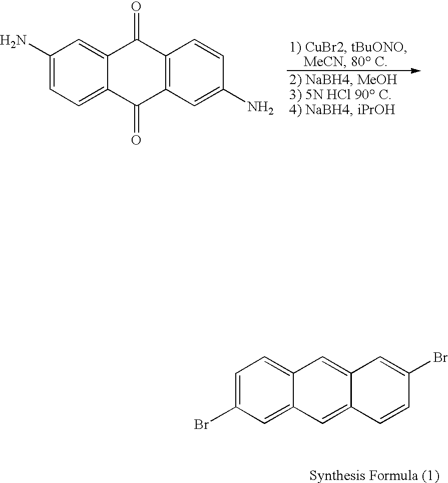

1) Cupric bromide (11.8 g) and tertiary butyl nitrite (7.4 g) were added to 500 ml of acetonitrile, and stirring was performed at 50° C. 2,6-Diaminoanthraquinone (5.7 g) was added in three portions into the reaction system, and stirring was performed at 50° C. for 8 hours. After the reaction was completed, the mixture was allowed to stand to cool to room temperature, and the solvent was removed by distillation under reduced pressure. The residue was washed with water and air-dried for two days to yield 8.5 g of 2,6-dibromoanthraquinone.

2) The resulting 2,6-dibromoanthraquinone (8.5 g) was suspended in 250 ml of methanol, and sodium borohydride (3.5 g) was added thereto in two portions under ice cooling. After stirring overnight at room temperature, the reaction solution was poured into water (500 ml), and the insoluble was filtered. The resulting solid was washed with water and air-dried to yield 4.4 g of a brown solid.

3) The resulting brown solid (4.4 g) was suspended in 5N hydrochloric acid, and stirring was performed at 70° C. for 6 hours. After standing to cool, the insoluble was filtered under reduced pressure and the resulting solid was washed with water and air-dried to yield 7.8 g of a green solid.

4) The resulting green solid (7.8 g) was suspended in 250 ml of isopropanol, and sodium borohydride (8.4 g) was added thereto in three portions under ice cooling. Subsequently, the temperature was raised to 50° C. and stirring was performed for 8 hours. After standing to cool, the reaction solution was poured into water (500 ml), and the insoluble was filtered. The resulting solid was washed with water and air-dried to yield a green solid.

The resulting green solid was washed with toluene to yield 5.0 g of 2,6-dibromoanthracene. The structure thereof was confirmed by 1H-NMR, 13C-NMR, and FD-MS.

The 2,6-dibromoanthracene (3.0 g) thus obtained, the aromatic borate ester (A1) (3.0 g) shown below, sodium hydroxide (0.5 g), and tetrakis(triphenylphosphino)palladium (0.05 g) were added to 100 ml of dry xylene, and a reaction was allowed to proceed for 3 hours at 100° C. in a nitrogen atmosphere.

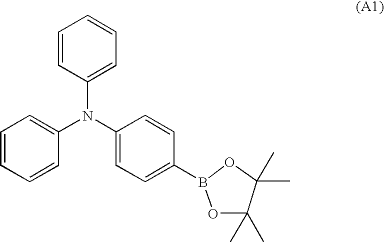

After the reaction was completed, an organic layer was separated, washing with water was performed twice and washing with saturated brine was performed once. After drying over anhydrous sodium sulfate, vacuum concentration was performed, followed by purification by silica gel column chromatography to yield 1.6 g of an intermediate (1) in yellow powder form. The resulting intermediate (1) was identified as the target compound through mass spectrometry (m/z 499).

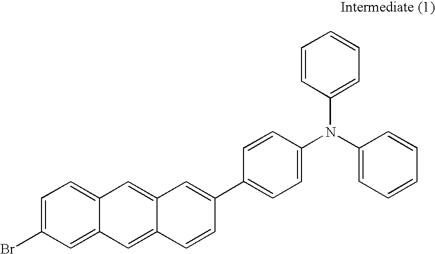

Subsequently, the resulting intermediate (1) (1.6 g), the aromatic borate ester (A2) (0.8 g) shown below, sodium hydroxide (0.2 g), and tetrakis(triphenylphosphino)palladium (0.05 g) were added to 100 ml of dry xylene, and a reaction was allowed to proceed for 6 hours at 100° C. in a nitrogen atmosphere.

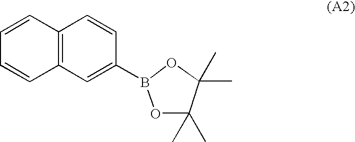

After the reaction was completed, an organic layer was separated, washing with water was performed twice and washing with saturated brine was performed once. After drying over anhydrous sodium sulfate, vacuum concentration was performed, followed by purification by silica gel column chromatography to yield 1.1 g of a compound (2) in yellow powder form. The resulting compound (2) was identified as the target compound through mass spectrometry (m/z 547).

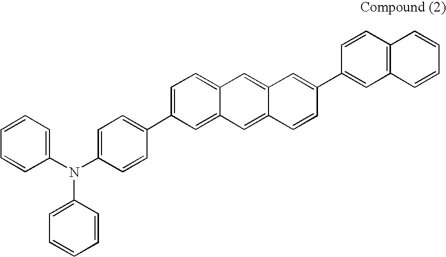

### Synthesis Example 2

### Synthesis of Compound (5)

A compound (5) (2.5 g) in yellow powder form was obtained as in Synthesis Example 1 except that the aromatic borate ester (A2) was replaced by the aromatic borate ester (A3) shown below. The resulting compound (5) was identified as the target compound through mass spectrometry (m/z 673).

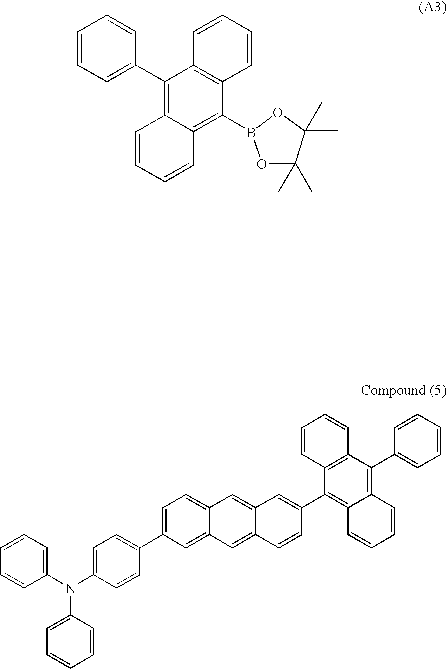

### Synthesis Example 3

### Synthesis of Compound (17)

A compound (17) (2.5 g) in yellow powder form was obtained as in Synthesis Example 1 except that the aromatic borate ester (A2) was replaced by the aromatic borate ester (A4) shown below. The resulting compound (17) was identified as the target compound through mass spectrometry (MS=573). FIG. 2 shows the fluorescence absorption spectrum of the resulting compound (17) in a dioxane solution.

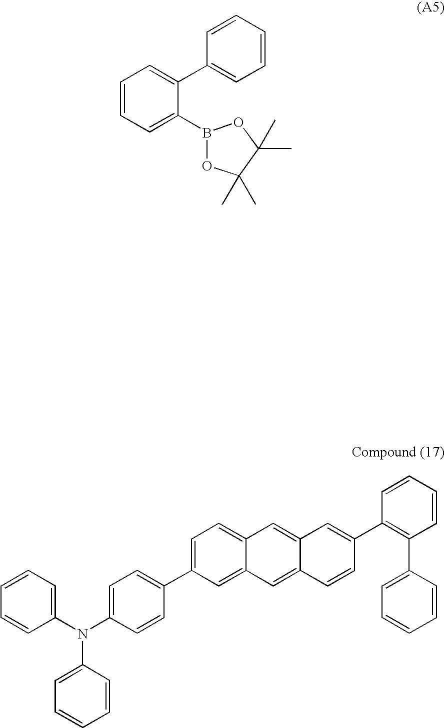

### Synthesis Example 4

### Synthesis of Compound (23)

Synthesis of Intermediate (2):

An intermediate (2) (2.0 g) in yellow powder form was obtained as in the synthesis procedure of the intermediate (1) in Synthesis Example 1 except that the aromatic borate ester (A1) was replaced by the aromatic borate ester (A5) shown below. The resulting intermediate (2) was identified as the target compound through mass spectrometry (m/z 447).

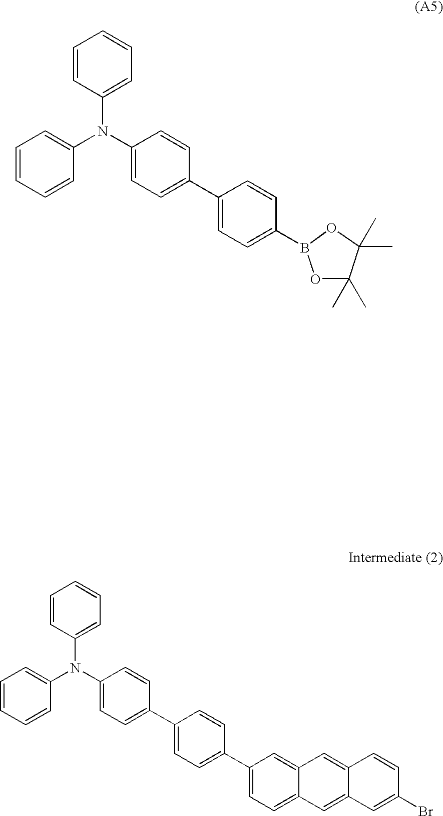

Subsequently, a compound (23) (1.7 g) in yellow powder form was obtained as in Synthesis Example 1 except that the intermediate (1) was replaced by the intermediate (2) and the aromatic borate ester (A2) was replaced by the aromatic borate ester (A4) shown below. The resulting compound (23) was identified as the target compound through mass spectrometry (m/z 649).

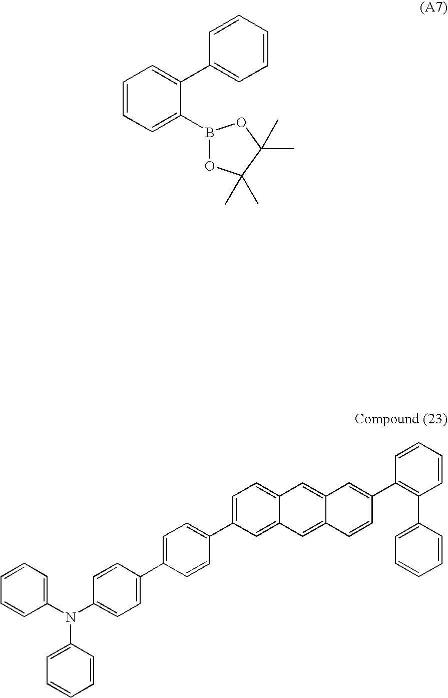

### Example 1

Using the compound (2) obtained in Synthesis Example 1, an organic electroluminescent device of a transmission type (refer to FIG. 1) was fabricated as described below.

An ITO transparent electrode (anode) was formed, as a lower electrode 4, with a thickness of 190 nm on a glass substrate 2 to produce an ITO substrate, and ultrasonic cleaning was performed with a neutral detergent, acetone, and ethanol. After the ITO substrate was dried, UV/ozone treatment was further performed for 10 minutes. Subsequently, the ITO substrate was fixed on a substrate holder of a deposition apparatus, and then the pressure of the deposition chamber was decreased to 1.4×10⁻⁴ Pa.

First, the N,N′-bis(1-naphthyl)-N,N′-diphenyl[1,1′-biphenyl]-4,4′-diamine (NPB) shown below was evaporated with a thickness of 65 nm on the ITO transparent electrode at an evaporation rate of 0.2 nm/sec to form a hole injection/transport layer 501.

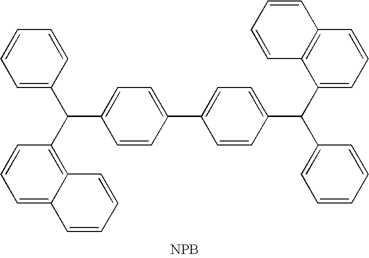

Subsequently, using the 9,10-di(2-naphthyl)anthracene (ADN) shown below as a host and the compound (2) shown below as a guest, coevaporation was performed from separate evaporation sources at a total evaporation rate of about 0.2 nm/sec with a thickness of 35 nm to form a luminescent layer 503, the guest concentration being 10% by volume.

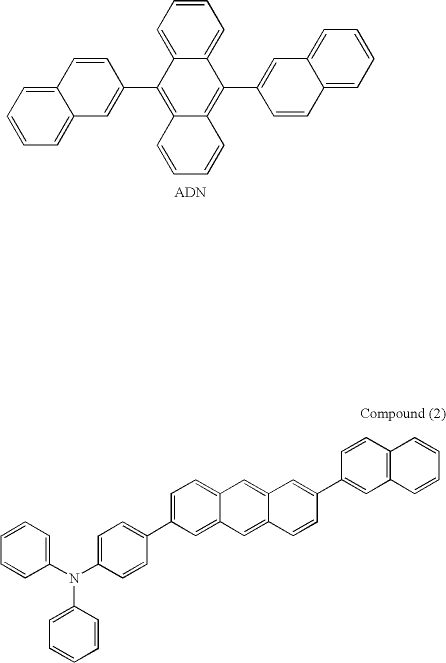

Subsequently, the Alq3 shown below was evaporated with a thickness of 18 nm at an evaporation rate of 0.2 nm/sec to form an electron-transport layer 505. Lithium fluoride (LiF) was evaporated thereon with a thickness of 0.1 nm, and furthermore, magnesium and silver were coevaporated at an evaporation rate of about 0.4 nm/sec with a thickness of 70 nm (atomic ratio Mg:Ag=95:5) to form a cathode (upper electrode 6). Thereby, an organic electroluminescent device 3 of a transmission type in which emitted light was extracted from the lower electrode 4 side was fabricated.

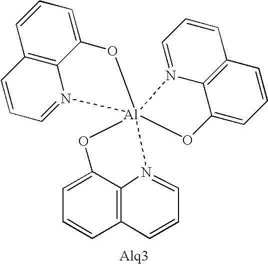

When the resulting organic electroluminescent device was driven with a direct current at a current density of 25 mA/cm2, the drive voltage (a) was 5.9 V, the luminous efficiency was 6.1 cd/A, and the power efficiency was 2.1 lm/W. Furthermore, blue luminescence was confirmed [luminescent luminance (b)=1,100 cd/m2, luminescence peak (c)=461 nm]. Furthermore, when this electroluminescent device was driven with a constant current at an initial luminance of 1,500 cd/m2, the half-lifetime (d) (i.e., time elapsed before the luminance of the electroluminescent device decreases to half its initial value) was 1,250 hours.

### Examples 2 to 11

Organic electroluminescent devices 3 of a transmission type were fabricated as in Example 1 except that the compound (5) and others shown as the guest material in Table 1 below were used instead of the compound (2) in the luminescent layer 503. Note that the guest concentration in each of the luminescent layers 503 was 10% by volume.

TABLE 1

| | Luminescent | Drive | Luminescent | | Half-lifetime |
|---|---|---|---|---|---|
| | layer | voltage (a) | luminance (b) | Luminescent | (d) |
| | Guest material | (V) | (cd/m2) | color (c) | (hour) |
| Example 1 | Compound (2) | 5.9 | 1,100 | Blue | 1,250 |
| Example 2 | Compound (5) | 5.5 | 1,210 | Blue | 1,350 |
| Example 3 | Compound (8) | 5.4 | 1,260 | Blue | 1,370 |
| Example 4 | Compound (9) | 6.1 | 990 | Blue | 1,200 |
| Example 5 | Compound (15) | 5.6 | 880 | Blue | 1,050 |
| Example 6 | Compound (17) | 6.1 | 1,030 | Blue | 1,250 |
| Example 7 | Compound (22) | 5.8 | 1,100 | Blue | 1,250 |
| Example 8 | Compound (23) | 6.1 | 990 | Blue | 1,200 |
| Example 9 | Compound (29) | 5.1 | 1,050 | Blue | 1,300 |
| Example 10 | Compound (41) | 5.6 | 1,080 | Blue | 1,250 |
| Example 11 | Compound (42) | 6.0 | 870 | Blue | 1,000 |
| Comparative | Compound (B1) | 5.9 | 1,050 | Green | 1,200 |
| Example 1 | | | | | |
| Comparative | BCzVBi 5% | 6.45 | 850 | Blue | 390 |
| Example 2 | | | | | |

With respect to the organic electroluminescent devices fabricated as described above in Examples 2 to 11, the measurement results (a) to (d) obtained as in Example 1 are also shown in Table 1.

### Comparative Example 1

An organic electroluminescent device was fabricated as in Example 1 except that the compound (B1) shown below was used at a guest concentration of 10% by volume instead of the guest composed of the compound (2) as the anthracene derivative in the luminescent layer 503.

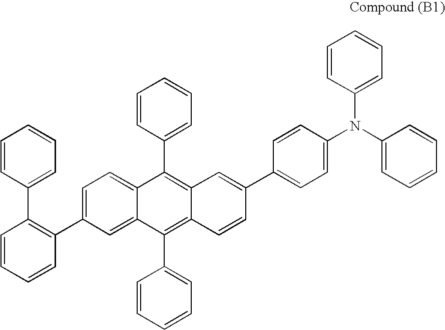

When the resulting organic electroluminescent device was driven with a direct current at a current density of 25 mA/cm2, the drive voltage (a) was 5.9 V, the luminous efficiency was 6.1 cd/A, and the power efficiency was 2.1 lm/W. Furthermore, green luminescence was confirmed [luminescent luminance (b): 1,050 cd/m2, luminescence peaks (c): 485 nm and 539 nm], and blue luminescence was not obtained. Furthermore, when this electroluminescent device was driven with a constant current at an initial luminance of 1,500 cd/m2, the half-lifetime (d) (i.e., time elapsed before the luminance of the electroluminescent device decreases to half its initial value) was 1,200 hours.

### Comparative Example 2

An organic electroluminescent device was fabricated as in Example 1 except that the BCzVBi shown below, which is described as a guest material for blue luminescence in Non-Patent Document 2, was used instead of the guest composed of the compound (2) as the anthracene derivative in the luminescent layer 503. The guest concentration was set at 5% by volume.

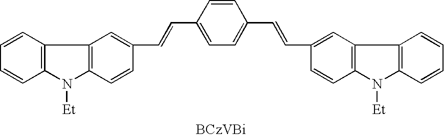

With respect to the organic electroluminescent device thus fabricated in Comparative Example 2, the measurement results (a) to (d) obtained as in Example 1 are also shown in Table 1.

As is evident from Table 1, in the organic electroluminescent devices in Examples 1 to 11 using, as the luminescent material, the anthracene derivatives [compound (2) and the like] according to the embodiments of the present invention, in which the 9,10 position in the anthracene skeleton is alkyl-substituted, alkoxy-substituted, or unsubstituted, blue luminescence can be obtained. Furthermore, the luminescent luminance exceeds 800 cd/m2, and the half-lifetime exceeds 1,000 hours.

In contrast, in the organic electroluminescent device in Comparative Example 1 using, as the luminescent material, the anthracene derivative in which the 9,10 position in the anthracene skeleton is aryl-substituted, the luminescent color is green and blue luminescence is not obtained. In the organic electroluminescent device in Comparative Example 2 using BCzVBi as the luminescent material, although blue luminescence is obtained, the half-lifetime is particularly short at 390 hours.

As described above, it has been confirmed that the anthracene derivative according to any of the embodiments of the present invention in which the 9,10 position in the anthracene skeleton is alkyl-substituted, alkoxy-substituted, or unsubstituted is a material having excellent luminous efficiency and life characteristics as a blue luminescent material in organic electroluminescent devices.

### Example 12

In Example 12, an organic electroluminescent device of a top emission type was fabricated.

An ITO transparent electrode (anode) with a thickness of 11 nm was formed on an Ag alloy layer with a thickness of 190 nm, as a lower electrode 4, on a glass substrate 2, and ultrasonic cleaning was performed with a neutral detergent, acetone, and ethanol. After drying, UV/ozone treatment was further performed for 10 minutes. Subsequently, the substrate was fixed on a substrate holder of a deposition apparatus, and then the pressure of the deposition chamber was decreased to 1×10⁻⁶ Torr.

First, the NPB described above was evaporated with a thickness of 24 nm on the ITO transparent electrode at an evaporation rate of 0.2 nm/sec to form a hole injection/transport layer 501. Subsequently, using the ADN described above as a host and the compound (17) as a guest, coevaporation was performed from separate evaporation sources at a total evaporation rate of about 0.2 nm/sec with a thickness of 35 nm to form a luminescent layer 503, the guest concentration being 10% by volume. Subsequently, the Alq3 described above was evaporated with a thickness of 18 nm at an evaporation rate of 0.2 nm/sec to form an electron-transport layer 505. Lithium fluoride (LiF) was evaporated thereon with a thickness of 0.1 nm, and furthermore, magnesium and silver were coevaporated at an evaporation rate of about 0.4 nm/sec with a thickness of 12 nm (atomic ratio Mg:Ag=95:5) to form a cathode (upper electrode 6). Thereby, an organic electroluminescent device 3 of a top emission type in which emitted light was extracted from the upper electrode 6 side was fabricated.

When the resulting organic electroluminescent device was driven with a direct current at a current density of 25 mA/cm2, the drive voltage (a) was 4.6 V, the luminous efficiency was 2.0 cd/A, and the power efficiency was 2.1 lm/W. Furthermore, blue luminescence was confirmed [luminescent luminance (b)=787 cd/m2, luminescence peak (c)=461 nm]. As a result, it has been confirmed that even in an organic electroluminescent device of a top emission type, by using, as the luminescent material, the anthracene derivative according to the embodiment of the present invention in which the 9,10 position in the anthracene skeleton is alkyl-substituted, alkoxy-substituted, or unsubstituted, blue luminescence can be obtained.

### Examples 13 to 17

Organic electroluminescent devices 3 of a transmission type were fabricated as in Example 1 except that the concentration of the guest composed of the compound (2) as the anthracene derivative in the luminescent layer 503 was respectively set at 1% by volume, 5% by volume, 10% by volume, 20% by volume, and 40% by volume.

With respect to the organic electroluminescent devices fabricated as described above, the drive voltage (a), the luminescent luminance (b), the luminescent color (c), and the half-lifetime (d) measured as in Example 1 are shown in Table 2 below.

TABLE 2

| | Luminescent | | | | |
|---|---|---|---|---|---|
| | layer | Drive | Luminescent | | Half-lifetime |
| | Concentration | voltage (a) | luminance (b) | Luminescent | (d) |
| | of compound (2) | (V) | (cd/m2) | color (c) | (hour) |
| Example 13 | 1% | 6.6 | 700 | Blue | 830 |
| Example 14 | 5% | 6.4 | 930 | Blue | 1,100 |
| Example 15 | 10% | 5.9 | 1,100 | Blue | 1,250 |
| Example 16 | 20% | 5.5 | 900 | Blue | 1,030 |
| Example 17 | 40% | 5.2 | 790 | Blue | 870 |

As is evident from Table 2, by setting the concentration of the anthracene derivative in the luminescent layer 503 to be 1% by volume or more and less than 40% by volume, it is possible to maintain high values with respect to the luminescent luminance (b) and the half-lifetime (d). Furthermore, by setting the concentration preferably at 1% to 20% by volume, and more preferably at 1% to 10% by volume, the luminescent luminance (b) and the half-lifetime (d) can be further increased.

### Synthesis Example 5

### Synthesis of Compound (45)

First, 2,6-dibromoanthracene was synthesized according to the synthesis formula (1) described above.

The resulting 2,6-dibromoanthracene (3.0 g), the aromatic borate ester (A1) (5.8 g) shown below, sodium hydroxide (1.5 g), tetrakis(triphenylphosphino)palladium (2.0 g) were added to 200 ml of dry xylene, and a reaction was allowed to proceed for 6 hours at 100° C.

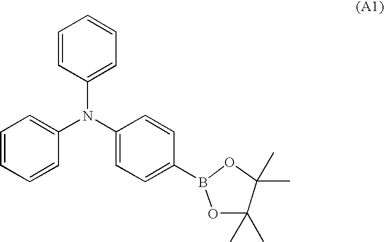

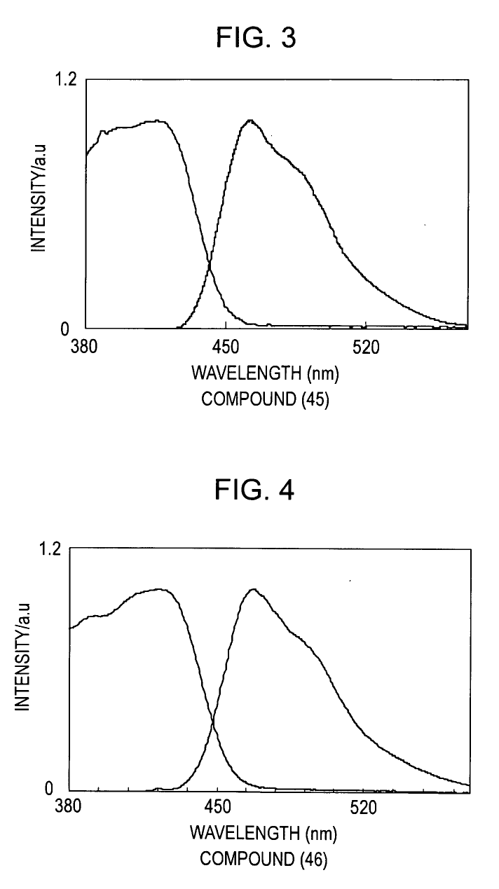

After the reaction was completed, the resulting precipitate was filtered, washed with water, and washed with hot acetone suspension to yield 2.8 g of a compound (45) in yellow powder form. The structure thereof was confirmed by 1H-NMR, 13C-NMR, and FD-MS. The resulting compound (45) was identified as the target compound through FD-MS (m/z 664). FIG. 3 shows the fluorescence absorption spectrum of the resulting compound (45) in a dioxane solution.

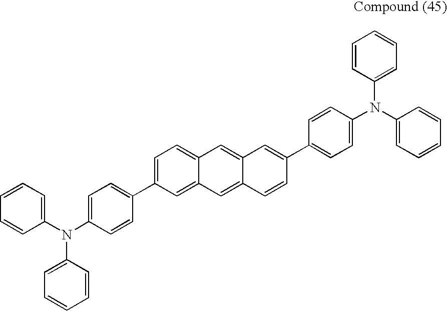

### Synthesis Example 6

### Synthesis of Compound (46)

A compound (46) (2.5 g) in yellow powder form was obtained as in Synthesis Example 5 except that the aromatic borate ester (A1) was replaced by the aromatic borate ester (A6) shown below. The structure thereof was confirmed by 1H-NMR, 13C-NMR, and FD-MS. The resulting compound (46) was identified as the target compound through FD-MS (m/z 804). FIG. 4 shows the fluorescence absorption spectrum of the resulting compound (46) in a dioxane solution.

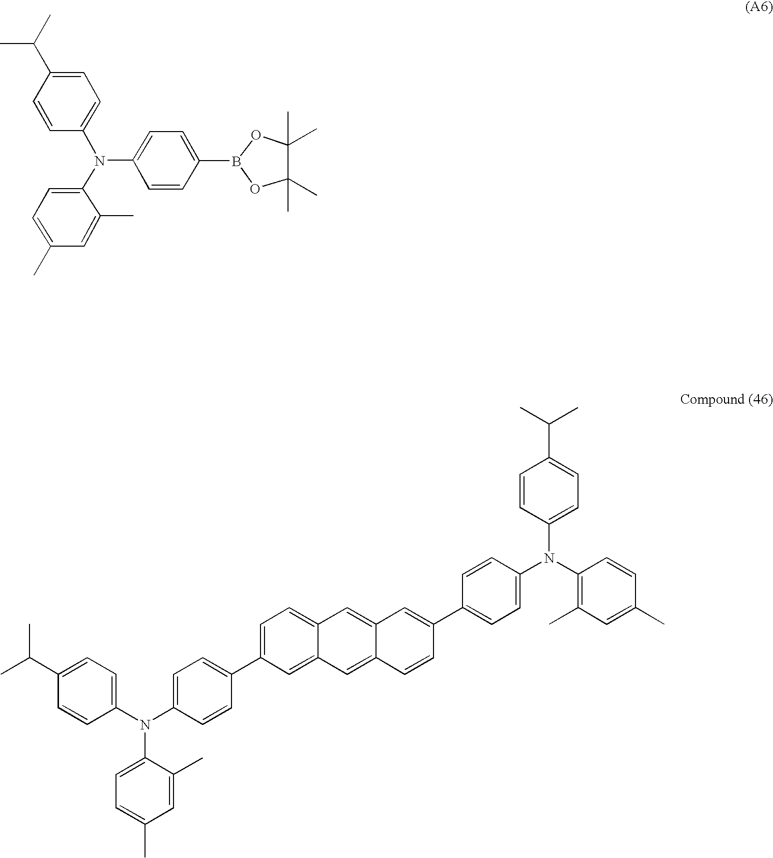

### Synthesis Example 7

### Synthesis of Compound (49)

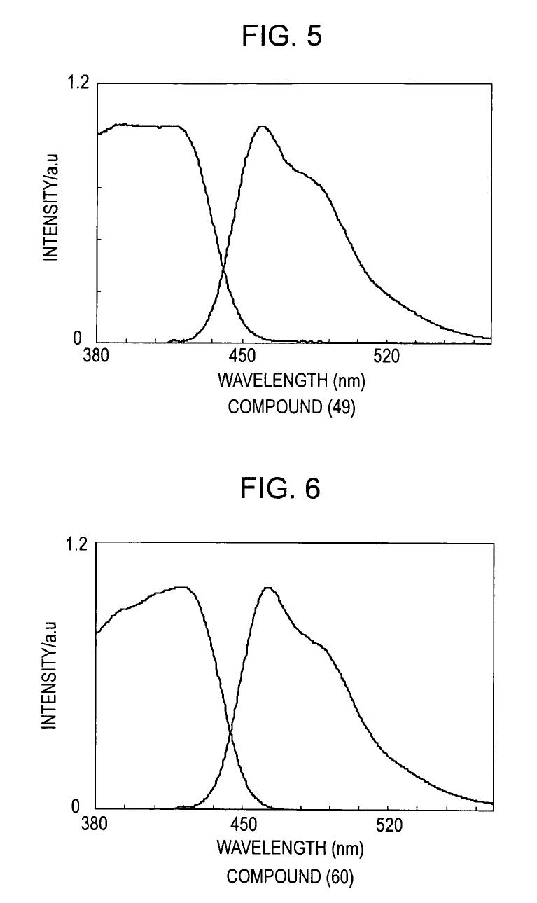

A compound (49) [2,6-bis{4-(N-(1-naphthyl)-N-phenylamino)phenyl}anthracene] (3.4 g) in yellow powder form was obtained as in Synthesis Example 5 except that the aromatic borate ester (A1) was replaced by the aromatic borate ester (A7) shown below. The structure thereof was confirmed by 1H-NMR, 13C-NMR, and FD-MS. The resulting compound (49) was identified as the target compound through FD-MS (m/z 764). FIG. 5 shows the fluorescence absorption spectrum of the resulting compound (49) in a dioxane solution.

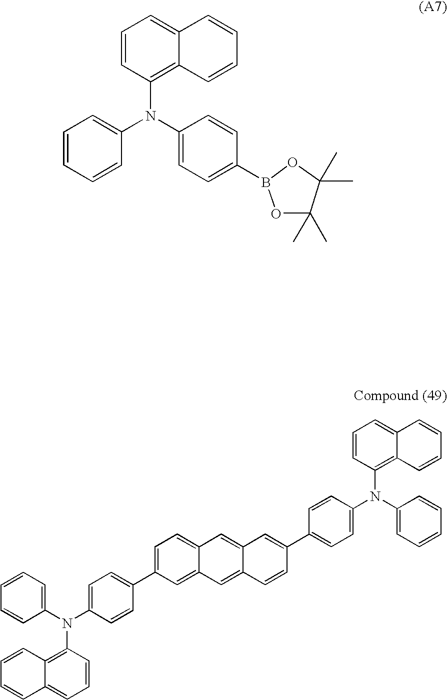

### Synthesis Example 8

### Synthesis of Compound (53)

A compound (53) [2,6-bis{3-(N,N-diphenylamino)phenyl)phenyl}anthracene] (2.5 g) in yellow powder form was obtained as in Synthesis Example 5 except that the aromatic borate ester (A1) was replaced by the aromatic borate ester (A8) shown below. The structure thereof was confirmed by 1H-NMR, 13C-NMR, and FD-MS. The resulting compound (53) was identified as the target compound through FD-MS (m/z 664).

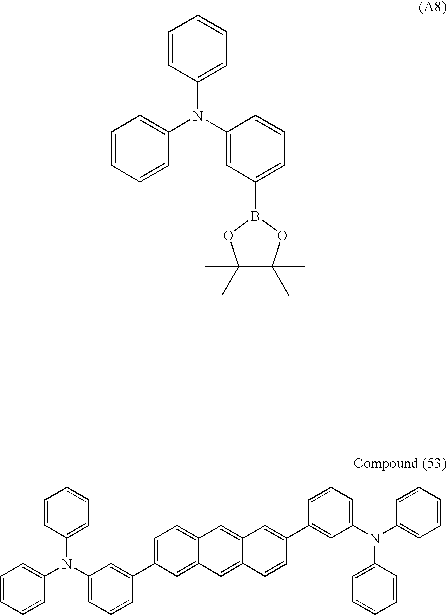

### Synthesis Example 9

### Synthesis of Compound (60)

A compound (60) (2.5 g) in yellow powder form was obtained as in Synthesis Example 5 except that the aromatic borate ester (A1) was replaced by the aromatic borate ester (A9) shown below. The structure thereof was confirmed by 1H-NMR, 13C-NMR, and FD-MS. The resulting compound (60) was identified as the target compound through FD-MS (m/z 816). FIG. 6 shows the fluorescence absorption spectrum of the resulting compound (60) in a dioxane solution.

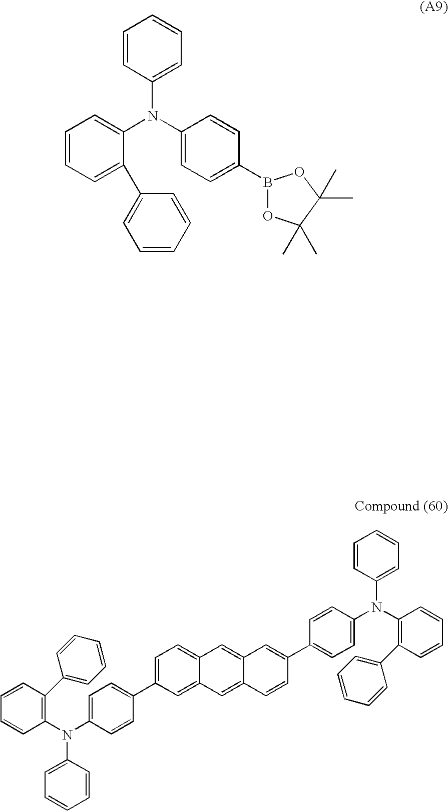

### Synthesis Example 10

### Synthesis of Compound (73)

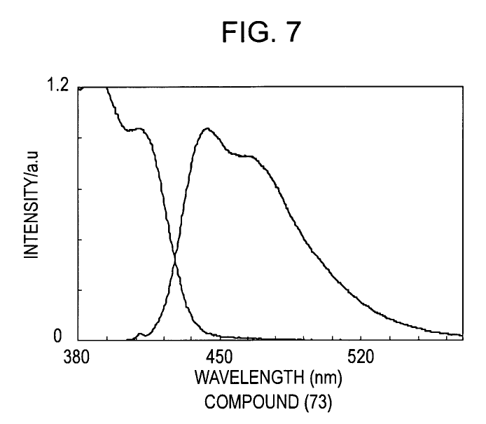

A compound (73) (2.5 g) in yellow powder form was obtained as in Synthesis Example 5 except that the aromatic borate ester (A1) was replaced by the aromatic borate ester (A5) shown below. The structure thereof was confirmed by 1H-NMR, 13C-NMR, and FD-MS. The resulting compound (73) was identified as the target compound through FD-MS (m/z 816). FIG. 7 shows the fluorescence absorption spectrum of the resulting compound (73) in a dioxane solution.

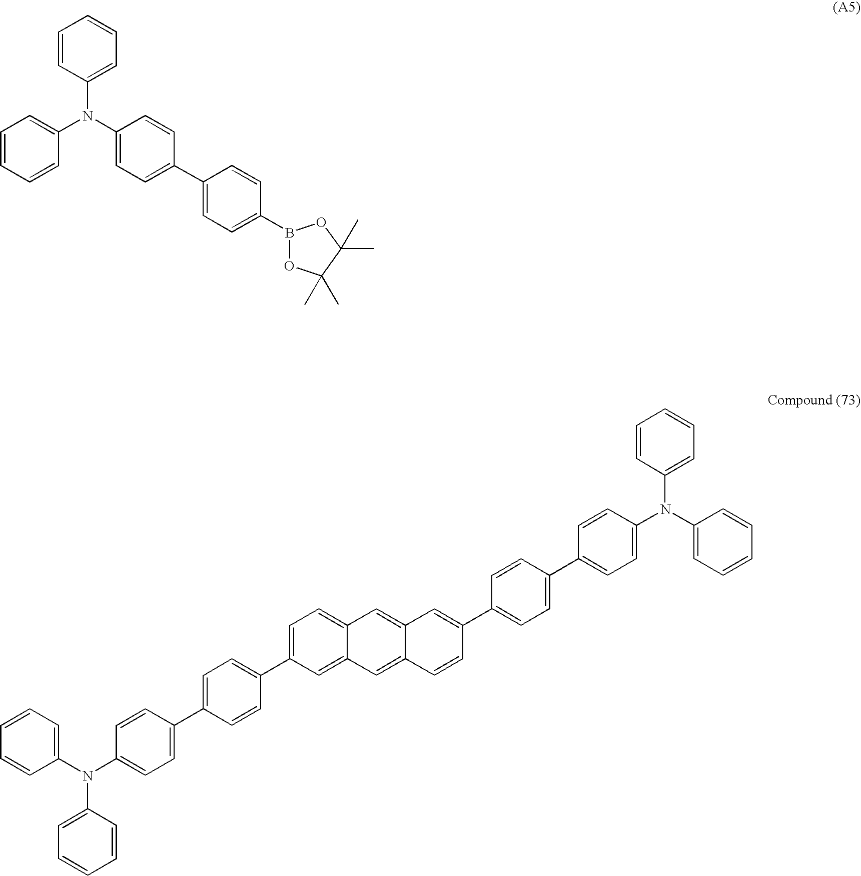

### Example 18

Using the compound (45) obtained in Synthesis Example 5, an organic electroluminescent device of a transmission type (refer to FIG. 1) was fabricated as in the examples described above.

When the resulting organic electroluminescent device was driven with a direct current at a current density of 25 mA/cm2, the drive voltage (a) was 5.7 V, the luminous efficiency was 6.4 cd/A, and the power efficiency was 3.5 lm/W. Furthermore, blue luminescence was confirmed [luminescent luminance (b)=1,610 cd/m2, luminescence peak (c)=468 nm]. Furthermore, when this electroluminescent device was driven with a constant current at an initial luminance of 1,500 cd/m2, the half-lifetime (d) (i.e., time elapsed before the luminance of the electroluminescent device decreases to half its initial value) was 1,800 hours.

### Examples 19 to 28

Organic electroluminescent devices 3 of a transmission type were fabricated as in Example 18 except that the compound (46) and others shown as the guest material in Table 3 below were used instead of the compound (45) in the luminescent layer 503. Note that the guest concentration in each of the luminescent layers 503 was 10% by volume.

TABLE 3

| | Luminescent | Drive | Luminescent | | Half-lifetime |
|---|---|---|---|---|---|
| | layer | voltage (a) | luminance (b) | Luminescent | (d) |
| | Guest material | (V) | (cd/m2) | color (c) | (hour) |
| Example 18 | Compound (45) | 5.7 | 1,610 | Blue | 1,800 |
| Example 19 | Compound (46) | 5.5 | 1,595 | Blue | 1,750 |
| Example 20 | Compound (49) | 5.9 | 1,550 | Blue | 1,750 |
| Example 21 | Compound (53) | 5.3 | 1,370 | Blue | 1,450 |
| Example 22 | Compound (60) | 5.6 | 1,615 | Blue | 1,800 |
| Example 23 | Compound (63) | 5.1 | 1,370 | Blue | 1,350 |
| Example 24 | Compound (65) | 5.3 | 1,410 | Blue | 1,400 |
| Example 25 | Compound (73) | 5.0 | 1,320 | Blue | 1,300 |
| Example 26 | Compound (85) | 5.0 | 1,250 | Blue | 1,250 |
| Example 27 | Compound (89) | 5.6 | 1,615 | Blue | 1,800 |
| Example 28 | Compound (91) | 5.8 | 1,330 | Blue | 1,250 |
| Comparative | Compound (B2) | 5.9 | 1,520 | Green | 1,700 |
| Example 3 | | | | | |
| Comparative | BCzVBi 5% | 6.5 | 850 | Blue | 390 |
| Example 4 | | | | | |

With respect to the organic electroluminescent devices fabricated as described above in Examples 19 to 28, the measurement results (a) to (d) obtained as in Example 18 are also shown in Table 3.

### Comparative Example 3

An organic electroluminescent device was fabricated as in Example 18 except that the compound (B2) shown below was used at a guest concentration of 10% by volume instead of the guest composed of the compound (45) as the anthracene derivative in the luminescent layer 503.

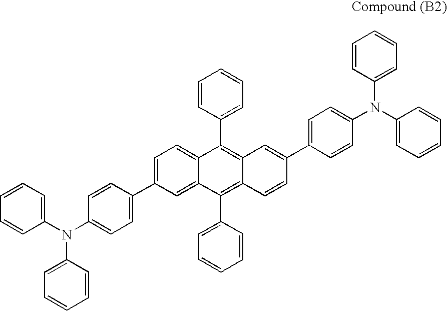

When the resulting organic electroluminescent device was driven with a direct current at a current density of 25 mA/cm2, the drive voltage (a) was 5.9 V, the luminous efficiency was 6.1 cd/A, and the power efficiency was 3.2 lm/W. Furthermore, green luminescence was confirmed [luminescent luminance (b): 1,520 cd/m2, luminescence peaks (c): 482 nm and 532 nm], and blue luminescence was not obtained. Furthermore, when this electroluminescent device was driven with a constant current at an initial luminance of 1,500 cd/m2, the half-lifetime (d) (i.e., time elapsed before the luminance of the electroluminescent device decreases to half its initial value) was 1,700 hours.

### Comparative Example 4

An organic electroluminescent device was fabricated as in Example 18 except that the BCzVBi shown below, which is described as a guest material for blue luminescence in Non-Patent Document 2, was used instead of the guest composed of the compound (45) as the anthracene derivative in the luminescent layer 503. The guest concentration was set at 5% by volume.

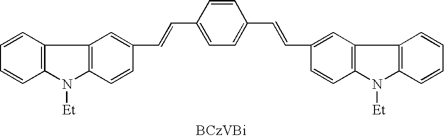

With respect to the organic electroluminescent device thus fabricated in Comparative Example 4, the measurement results (a) to (d) obtained as in Example 18 are also shown in Table 3.

As is evident from Table 3, in the organic electroluminescent devices in Examples 18 to 28 using, as the luminescent material, the anthracene derivatives [compound (45) and the like] according to the embodiments of the present invention, in which the 9,10 position in the anthracene skeleton is alkyl-substituted, alkoxy-substituted, or unsubstituted, blue luminescence can be obtained. Furthermore, the luminescent luminance exceeds 1,000 cd/m2, and the half-lifetime exceeds 1,200 hours.

In contrast, in the organic electroluminescent device in Comparative Example 3 using, as the luminescent material, the anthracene derivative in which the 9,10 position in the anthracene skeleton is aryl-substituted, the luminescent color is green and blue luminescence is not obtained. In the organic electroluminescent device in Comparative Example 4 using BCzVBi as the luminescent material, although blue luminescence is obtained, the half-lifetime is particularly short at 390 hours.

As described above, it has been confirmed that the anthracene derivative according to any of the embodiments of the present invention in which the 9,10 position in the anthracene skeleton is alkyl-substituted, alkoxy-substituted, or unsubstituted is a material having excellent luminous efficiency and life characteristics as a blue luminescent material in organic electroluminescent devices.

### Example 29

In Example 29, an organic electroluminescent device of a top emission type was fabricated.

When the resulting organic electroluminescent device was driven with a direct current at a current density of 25 mA/cm2, the drive voltage (a) was 4.6 V, the luminous efficiency was 3.1 cd/A, and the power efficiency was 2.1 lm/W. Furthermore, blue luminescence was confirmed [luminescent luminance (b)=763 cd/m2, luminescence peak (c)=467 nm]. As a result, it has been confirmed that even in an organic electroluminescent device of a top emission type, by using, as the luminescent material, the anthracene derivative according to the embodiment of the present invention in which the 9,10 position in the anthracene skeleton is alkyl-substituted, alkoxy-substituted, or unsubstituted, blue luminescence can be obtained.

### Examples 30 to 34

Organic electroluminescent devices 3 of a transmission type were fabricated as in Example 18 except that the concentration of the guest composed of the compound (45) as the anthracene derivative in the luminescent layer 503 was respectively set at 1% by volume, 5% by volume, 10% by volume, 20% by volume, and 40% by volume.

With respect to the organic electroluminescent devices fabricated as described above, the drive voltage (a), the luminescent luminance (b), the luminescent color (c), and the half-lifetime (d) measured as in Example 18 are shown in Table 4 below.

TABLE 4

| | Luminescent | | | | |
|---|---|---|---|---|---|
| | layer | | | | |
| | Concentration | Drive | Luminescent | | Half-lifetime |
| | of compound | voltage (a) | luminance (b) | Luminescent | (d) |
| | (45) | (V) | (cd/m2) | color (c) | (hour) |
| Example 30 | 1% | 5.8 | 1,140 | Blue | 1,270 |
| Example 31 | 5% | 5.5 | 1,520 | Blue | 1,710 |
| Example 32 | 10% | 5.7 | 1,610 | Blue | 1,800 |
| Example 33 | 20% | 5.1 | 1,080 | Blue | 1,650 |
| Example 34 | 40% | 4.8 | 850 | Blue | 1,300 |

As is evident from Table 4, by setting the concentration of the anthracene derivative in the luminescent layer 503 to be 1% by volume or more and less than 40% by volume, it is possible to maintain high values with respect to the luminescent luminance (b) and the half-lifetime (d). Furthermore, by setting the concentration preferably at 1% to 20% by volume, and more preferably at 1% to 10% by volume, the luminescent luminance (b) and the half-lifetime (d) can be further increased.

It should be understood by those skilled in the art that various modifications, combinations, sub-combinations and alterations may occur depending on design requirements and other factors insofar as they are within the scope of the appended claims or the equivalents thereof.

What is claimed is:

1. An anthracene derivative represented by general formula (1): 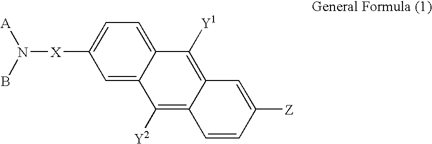
wherein X represents a substituted or unsubstituted arylene group having 6 to 28 carbon atoms, or a substituted or unsubstituted divalent heterocyclic group having 5 to 21 carbon atoms;
A and B each independently represent a substituted or unsubstituted alkyl group having 1 to 20 carbon atoms, a substituted or unsubstituted aryl group having 6 to 28 carbon atoms, or a substituted or unsubstituted heterocyclic group having 5 to 21 carbon atoms, and A and B may be bonded together to form a ring;
Y¹ and Y² each independently represent a hydrogen atom, a substituted or unsubstituted alkyl group having 1 to 20 carbon atoms, or an alkoxy group having 1 to 20 carbon atoms; and
Z represents a substituted or unsubstituted aryl group having 6 to 30 carbon atoms, a substituted or unsubstituted heterocyclic group having 5 to 21 carbon atoms, a hydrogen atom, a substituted or unsubstituted alkyl group having 1 to 20 carbon atoms, or an alkoxy group having 1 to 20 carbon atoms.

2. The anthracene derivative according to claim 1, wherein in general formula (1), X represents a substituted or unsubstituted arylene group having 6 to 16 carbon atoms, or a substituted or unsubstituted divalent heterocyclic group having 5 to 13 carbon atoms;
A and B each independently represent a substituted or unsubstituted alkyl group having 1 to 20 carbon atoms, a substituted or unsubstituted aryl group having 6 to 16 carbon atoms, or a substituted or unsubstituted heterocyclic group having 5 to 17 carbon atoms, and A and B may be bonded together to form a ring; and
Z represents a substituted or unsubstituted aryl group having 6 to 30 carbon atoms, or a substituted or unsubstituted heterocyclic group having 5 to 21 carbon atoms.

3. An anthracene derivative represented by general formula (2): 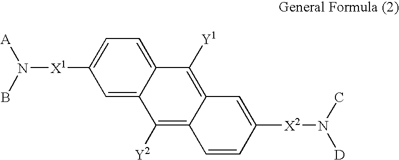
wherein X¹ and X² each independently represent a substituted or unsubstituted arylene group having 6 to 28 carbon atoms, or a substituted or unsubstituted divalent heterocyclic group having 5 to 21 carbon atoms;
A, B, C, and D each independently represent a substituted or unsubstituted alkyl group having 1 to 20 carbon atoms, a substituted or unsubstituted aryl group having 6 to 28 carbon atoms, or a substituted or unsubstituted heterocyclic group having 5 to 21 carbon atoms, and A and B may be bonded together to form a ring and/or C and D may be bonded together to form a ring; and
Y¹ and Y² each independently represent a hydrogen atom, a substituted or unsubstituted alkyl group having 1 to 20 carbon atoms, or an alkoxy group having 1 to 20 carbon atoms.

4. The anthracene derivative according to claim 3, wherein in general formula (2), X¹ and X² each independently represent a substituted or unsubstituted arylene group having 6 to 16 carbon atoms, or a substituted or unsubstituted divalent heterocyclic group having 5 to 13 carbon atoms; and
A, B, C, and D each independently represent a substituted or unsubstituted alkyl group having 1 to 20 carbon atoms, a substituted or unsubstituted aryl group having 6 to 16 carbon atoms, or a substituted or unsubstituted heterocyclic group having 5 to 17 carbon atoms, and A and B, and C and D each may be bonded together to form a ring.

5. The anthracene derivative according to claim 1 or 3, wherein in general formula (1) or (2), X, X¹, and X² each represent a substituted or unsubstituted phenylene group.

6. The anthracene derivative according to claim 1 or 3, wherein in general formula (1) or (2), Y¹ and Y² each represent a hydrogen atom.

7. An organic electroluminescent device comprising:
a pair of electrodes; and
an organic layer sandwiched between the pair of electrodes, the organic layer including at least a luminescent layer,
wherein the organic layer includes the anthracene derivative according to any one of claims 1 to 6.

8. The organic electroluminescent device according to claim 7, wherein the anthracene derivative is used as a material constituting the luminescent layer.

9. The organic electroluminescent device according to claim 8, wherein the anthracene derivative is used as a blue luminescent material.

10. The organic electroluminescent device according to claim 8 or 9, wherein the luminescent layer contains the anthracene derivative in an amount not exceeding 20% by volume.

11. The organic electroluminescent device according to claim 7, wherein the anthracene derivative is used as at least one material selected from a hole-injecting material, a hole-transporting material, and a doping material in the organic layer.

12. A display unit comprising:
a substrate; and
a plurality of organic electroluminescent devices arrayed on the substrate, each organic electroluminescent device including an organic layer sandwiched between an anode and a cathode, the organic layer including at least a luminescent layer,
wherein the organic electroluminescent devices include at least one organic electroluminescent device according to any one of claims 7 to 11.

13. The display unit according to claim 12, wherein the organic electroluminescent device according to any one of claims 7 to 11 is provided as a blue luminescent device in a part of a plurality of pixels.
